(19) United States
(12) Patent Application Publication
(10) Pub. No.: US 2014/0004204 A1
(43) Pub. Date: Jan. 2, 2014

(54) BIOCOMPATIBLE POLYACRYLATE COMPOSITIONS AND METHODS OF USE

(71) Applicant: University of South Florida, Tampa, FL (US)

(72) Inventors: KERRIANN ROBYN GREENHALGH, TAMPA, FL (US); EDWARD TUROS, WESLEY CHAPEL, FL (US)

(21) Appl. No.: 13/920,553

(22) Filed: Jun. 18, 2013

Related U.S. Application Data
(63) Continuation of application No. PCT/US2013/030848, filed on Mar. 13, 2013.
(60) Provisional application No. 61/666,564, filed on Jun. 29, 2012.

(51) Int. Cl. A61K 47/32

(52) U.S. Cl. CPC A61K 47/32
USPC 424/497; 514/772.6

(57) ABSTRACT

A biocompatible polymer material is described that exhibits mechanical and physical properties that are fundamental to many medical devices and treatment of many medical diseases and disorders. The material is composed of a combination of acrylate monomers polymerized via a microemulsion polymerization. Multiple applications of the polymer material are presented.

### CROSS-REFERENCE TO RELATED APPLICATIONS

The present application is a continuation of International Application No. PCT/US2013/030848, filed Mar. 13, 2013, which claims the benefit of U.S. Provisional Application Ser. No. 61/666,564, filed Jun. 29, 2012, each of which is hereby incorporated by reference herein in its entirety, including any figures, tables, nucleic acid sequences, amino acid sequences, and drawings.

### GOVERNMENT SUPPORT

This invention was made with government support under grant number R01 AI 51351 awarded by the National Institutes of Health and grant number NSF 0419903 awarded by the National Science Foundation. The government has certain rights in the invention.

### BACKGROUND OF THE INVENTION

A wound is defined as an injury, usually involving division or rupture of tissue in the integument or mucous membrane, due to external forces, mechanical insult, or disease. A wound can be caused by pressure, puncture, heat or friction.⁴⁷ Examples of these wounds include pressure ulcers, bedsores, scrapes and burns. There are many different varieties of wounds and they often require different methods of treatment. Some are shallow, producing low exudate, while others may be deep wounds and produce high amounts of exudate.

There are two significant elements to wound healing; repair and regeneration. Wound repair results from connective tissue replacing lost cells. This leads to scar formation. Wound regeneration occurs when lost cells and tissues are replaced by cells of the same type. Wound dressings promote this process. There are two classifications of wound dressings. They can either be a primary or a secondary dressing. A primary dressing is positioned directly onto the wound. It is the main source of support, protection, and absorption and serves as a mounting point for a secondary bandage. A secondary bandage is placed over the primary dressing and provides additional support, protection and absorption.

There are several desirable characteristics of wound dressings. They should protect the wound, keep it clean, and prevent infection. The wound dressing should be strong, inexpensive, absorbent, protective, and able to conform to the area it is placed in order to achieve these requirements.⁵⁶ An important characteristic of a bandage is to prevent infection while healing occurs. To prevent infection, antibiotics are often used, and in most cases must be administered in the hospital via intravenous administration due to limitations of the current topically applied antibiotics. In cases of chronic wounds which are not debilitating, patients are still required to be checked into hospitals for the IV antibiotic treatment, significantly increasing healthcare costs and inconvenience to patients. Antibiotics eliminate or inhibit the growth of microbes. Examples of antibiotics include penicillin, bacitracin, ciprofloxacin and vancomycin. Antibiotics used in conjunction with bandages enable the wound to heal with a much lower risk of infection.

There are a wide variety of wound dressings that are currently in use. These include gauze, tulles, hydrocolloids, alginates, foams, and hydrogels, among others. Gauzes, one of the most commonly used dressings, are composed of a thin fabric with a loose open weave. Dressings composed of gauze, however, can stick to the wound surface and disrupt the wound bed when removed so it is used only on minor wounds or as secondary dressings mainly to absorb exudate. Tulle is very similar to gauze but uses a light and very fine netting. Unlike gauze, tulle does not stick to the wound surface. It is suitable for flat and shallow wounds and is very useful in patients with sensitive skin. Examples of tulle bandages include JELONET and PARANET.⁵⁰

Semi-permeable film bandages are acrylic coated sterile sheets of polyurethane. They are suitable for shallow wounds that do not produce much exudate and are transparent facilitating easy access for wound checks. Examples of these include OPSITE and TEGADERM bandages.⁵⁰

Hydrocolloids are composed of gelatin, elastomers, pectin, carboxymethylcellulose and adhesives that transform into a gel when moisture, in this case exudate, is absorbed. Depending on the type of hydrocolloid dressing chosen, it can be used on wounds with light to heavy exudate and sloughing or granulating wounds. It is most commonly found in self-adhesive pads but can be a paste, powder, or non-adhesive pad. Examples include DUODERM and TEGASORB dressings.⁵⁰

Polyurethane and or silicone foam bandages are designed to absorb large amounts of exudates. They maintain the moist and sealed environment for healing but are not as useful as hydrocolloids for wound debridement. As by the design to absorb large amounts of exudates, these foam bandages do not work well on low exudating wounds, as dryness and scabbing will be the result. Examples of these bandages include ALLEVYN and LYOFOAM.⁵⁰

Alginates are composed of calcium alginate. As the name suggests it is extracted from seaweed. When the dressing comes in contact with the wound the calcium contained is exchanged with sodium from the wound fluid and transforms the dressing into a gel. This type of bandage is good for exudating wounds but when used with low exudating wounds it will cause dryness and scabbing. Examples of alginates include KALTOSTAT and SORBSAN. Other types of bandages include hydrofiber and collagen bandages. Hydrofiber bandages are composed of a soft non-woven pad or ribbon made from sodium carboxymethycellulose fibers. When these fibers come into contact with wound exudate it turns into a gel. Hydrofiber bandages are able to absorb exudate and can be used in deep wounds. Collagen bandages promote the deposition of newly formed collagen into the wound bed. They come in pads, gels or powder form.⁵⁰

A hydrogel bandage is composed of a network of polymer chains that are dispersed in water. Hydrogels are superabsorbent as they contain over 99% water and natural or synthetic polymers and possess a degree of flexibility very similar to natural tissue. Hydrogels are either amorphous or available in sheet form. These two types of hydrogels are similar in composition in that they contain significant portions of water and smaller amounts of polymers and thickening agents (Mary Anne Crandall. Kalorama Information (2011). Wound Care Markets 2011). Amorphous gels are more effective in donating moisture to tissue but cannot be used in deep wounds and should only coat the surfaces of wound cavities, not fill the cavities, and should be filled subsequent with gauze or other secondary bandages. They are clear gels of varying viscosity and can be applied directly to the wound surface. Sheet hydrogels are also high in water content but are not as efficient at donating their water because it has been bound in a cross-linked polymer network, which gives it form (Mary Anne Crandall. Kalorama Information (2011). Wound Care Markets 2011). When used as scaffolds, hydrogels may contain human cells in order to repair tissue.⁵³ Hydrogel dressings have been proven effective in facilitating the repair of pressure ulcers, diabetic ulcers, and burns in addition to acute wounds such as cuts, scrapes and surgical wounds. The water content in a hydrogel can be widely adjusted so they can be moist, if desired, or more absorbent to enable the absorption of wound exudate. Hydrogels can adhere to the intact skin without sticking directly to the injury or wound bed and can possess a degree of flexibility that is very similar to natural tissue.⁵⁴

Liquid bandages are primarily comprised of polymers that are strongly adhesive and are applied to the skin via an alcohol or acetone solvent. A liquid bandage is a sterile device that is a liquid, gel, or powder and liquid combination used to protect minor cuts and skin abrasions from infection. The device is also often used as a topical skin protectant. Many liquid bandages are formed from acrylate polymers such as cyanoacrylate. Polyacrylates have been used since the 1960s as biomedical coatings on devices and surgical glues, and are considered nontoxic²⁶⁻³⁵; moreover, emulsified polyacrylates, likewise, have been studied as colloidal drug carriers and hydrogels.^{11-18,28,36-41}

There are a few compounds used on the market today that act as biocompatible glues or bandages. The main types are cyanoacrylates, fibrin sealants, collagen-based compounds, glutaraldehyde and gelatins. Cyanoacrylates are used in bandages such as Johnson and Johnson's SINGLE STEP™ liquid bandage. There are predominantly two types of cyanoacrylates that are used in liquid bandages, ethyl cyanoacrylate and butyl cyanoacrylate. Ethyl cyanoacrylate is the main ingredient in superglue. It is also used as a tissue adhesive in lieu of suture or staples for surgical and emergency closure of skin. Ethyl cyanoacrylate however has a few negative aspects; it breaks down under high heat and produces eye and lung irritating gaseous products. Butyl cyanoacrylate can be injected into the body and can be used as adhesives for lacerations of the skin and bleeding vascular structures. Butyl cyanoacrylate however has a sharp irritating odor and both versions are often the result of accidental skin adhesions and emergency room visits.

Some bandages on the market have compounds added to increase functionality, often times with negative effects. New Skin Antiseptic, for example, is a liquid bandage suspended in alcohol solutions to provide antiseptic qualities, but this causes the bandage to sting and burn patients. There have been several customer reviews on liquid bandages. A few examples of the positives of the products currently on the market include that it is a good idea for those who cannot or will not wear Band-Aids, it is inexpensive, and is conforms to all surfaces. However, there were several negatives which consisted of the smell, the films attract debris, they pull on the skin and do not move with the skin, they are not very durable, and they burned terribly. These factors deterred individuals from using the product and especially in the case of parents whose children literally ran when the product was opened.⁵⁵

Diabetic wounds are complex environments that are invariably difficult to treat. Due to the high occurrence of diabetes in America, diabetic microvascular skin ulcers have become a major health concern. Diabetes has created a large need in the wound care market; one that is still unfulfilled. The annual US surgical procedure volume for diabetic foot ulcers is approx. 800,000 and around 500,000 for venous leg ulcers. Chronic wounds present a unique challenge for any wound treatment product due to the extremely fragile environment, the inherently slow healing rate, and the heightened risk of infection. While a number of products have emerged in the recent years that are capable of covering these complex wounds, there has yet to be a product that is truly conformable, continuously maintains a balanced moist environment, address prolonged infection, and is non-disruptive to the healing process.

Neuropathic skin ulcers, also known as diabetic neuropathic ulcers, occur in people who have little or no sensation in their feet due to diabetic nerve damage. These skin ulcers develop at pressure points on the foot, such as on the heel, the great toe, or other spots that rub on footwear.

Diabetics are prone to ulcers due to neurologic and vascular complications. Peripheral neuropathy is often experienced by diabetics and causes an altered or complete loss of sensation in the foot and/or leg. Therefore, any cuts or trauma to the foot can go completely unnoticed for days or weeks in a patient with neuropathy and a diabetic with advanced neuropathy loses this sensation resulting in tissue ischemia and necrosis. A major issue in treatment of these ulcerations is that excess discharge must be absorbed and a moist wound environment must be maintained in order for any substantial healing to occur. Infection here is also a major concern, where amputation is often the end result due to the inability of the physician to effectively treat the infection within the wound bed. Infection in these complex wounds environments ultimately prolongs healing and causes damage to surrounding healthy tissue and the potential for sepsis, toxic shock syndrome (TSS) and death. The incidence of microbial infection is significantly increased when factors such as diabetic microvascular changes contribute to wound formation. Chronic vascular and diabetic ulcers often persist for many months, during which time microbial resistance to antibiotic therapies can easily occur. Typical treatment regimen for diabetic ulcers includes wound cleansing, aseptic surgical debridement, then application of a hydrogel dressing to the wound base, that is often covered by a foam dressing for heavy exudating wounds.

Many hydrocolloid/hydrogel products are currently on the market, including the 3M TegaSorb and Systagenix NuDerm, and the hydrogel products include AcryMed's FlexiGel, Systagenix NuGel and the recently approved silver-containing hydrogel from American Biotech Labs, Antibacterial Silver Wound Dressing Gel. Many of the hydrogel, as well as film products, have turned towards silver for their antimicrobial activity. The silver anti-infective area in wound care has been re-invented by numerous companies and still has yet to overcome the basic issues of cytotoxicity, discoloration, sensitization, and microbial resistance. An additional underlying downside to all of the aforementioned products is the need for secondary dressing coverage to prevent infection and to help trap the moisture delivered to the wounds.

### BRIEF SUMMARY OF THE INVENTION

The present invention concerns compositions having unique properties that make them ideal for skin care, tissue and wound care, drug delivery, device coatings, and other medical applications, and methods for their use. The compositions of the invention comprise an emulsion of nanoparticles and water, the nanoparticles comprising a copolymer of a base acrylate monomer and a supporting monomer, preferably polymerized via micro emulsion polymerization. These polymer materials are biocompatible and exhibit mechanical and physical properties that are fundamental to many medical applications and treatment of many diseases and disorders. Accordingly, the compositions of the invention may be made or adapted to form a medical device (human or veterinary medical device), or a component of a medical device, intended for contact with the body, such as a patch, wound dressing, bandage, or implant, or a layer or coating on a surface of such a device.

The unique polyacrylate formulations described herein provide a number of advantages over the major hydrocolloid and hydrogel competitors in the wound care market. When applied to a wound, a typical hydrogel hydrates the wound surface and softens necrotic tissue, allowing autolytic debridement. Patients often find hydrogels soothing on wounds, and are easy to use, non-adherent, and ideal for use on delicate tissue. However, some of the major drawbacks to the use of hydrogels are that they are non-absorptive, require subsequent coverage to prevent infection, and the majority of hydrogels, aside from the limited number of silver-containing hydrogel products, do not address infection. The compositions of the invention, which are also hydrogels, avoid all of the drawbacks that are well documented with the use of typical hydrogels. The compositions of the invention can be used with or without secondary bandages due to the inherent film formation process that protects wounds and blocks bacteria. When formulated as a film, for example, the composition of the invention is absorptive as well, and does not require dressing changes. Wound management can be significantly simplified with use of the invention.

The biocompatible compositions described herein may be applied as a liquid bandage. The compositions use acrylate monomers to form complex polymer chains in a water-based solution. The compositions of the invention lack the side effects of commercial liquid bandages, such as ethyl or butyl cyanoacrylate bandages. The compositions of the invention are suspended in water and thus do not sting, burn the patients, nor have an odor (unless desired), and can also be used on a much wider range of wounds in comparison with liquid or traditional bandages. The compositions of the invention absorb exudate, do not allow bacterial ingrowth, prevent scab and scar formation, and when removed do not irritate or disturb newly formed skin or granulation tissue. Along with these advantages, medication, antibiotics, and other compounds may be bound to the compositions. These include antibiotics, non-steroidal and steroidal anti-inflammatory agents, anti-fungals, painkillers, and other agents useful for skin care and therapeutic agents.³⁷ For example, the compositions may include nicotine. This enables the compositions to be used not only as medical material for wound repair but also as a drug delivery agent, such as a liquid nicotine patch. This enables a more flexible dosage of medication to be used with less expense to the consumer.

### BRIEF DESCRIPTION OF THE DRAWINGS

FIG. 1. Potential acrylation scheme for bacitracin.

FIG. 2. Nuclear magnetic resonance (NMR) spectra of polymyxin B sulfate dissolved in D₂O.

FIG. 3. NMR spectra of acrylated derivative of polymyxin B dissolved in D₂O.

FIGS. 4A-B. Two potential schemes for the acrylation of the amine sites of polymyxin B.

FIG. 5. Scheme for the acrylation of one of the carboxylic acids sites of bacitracin.

FIG. 6. Scheme for the acrylation of the amine sites of bacitracin.

FIG. 7. Scheme for the acrylation of neomycin.

FIG. 8. Scheme for the acrylation of thiabendazole.

FIG. 9A-C. Scheme for the acrylation of prednisone and H1 NMR of pure prednisone and prednisone acrylate, with chloroform-D as the solvent.

FIG. 10. Nanoparticle polyacrylate emulsion at 20% solid content.

FIG. 11. Atomic force microscopy (AFM) image of drug-free nanoparticle polyacrylate emulsion.

FIG. 12A-C. AFM image of polyacrylate emulsion containing pencillin G, ciprofloxacin and beta-lactams (FIGS. 12A and 12B) and SEM of beta-lactam bound ethyl acrylate particles (FIG. 12C).

FIG. 13. Images of a butyl acrylate-styrene polymer film (without drugs or additives) before and during mechanical testing. Initial film length placed between the clamps is approximately 10 mm and the film is stretch to 100 mm, approximately a 1000% deformation.

FIG. 14. Fourier transform infrared spectrometry (FTIR) spectra of butyl acrylate-styrene and butyl acrylate-methyl methacrylate films.

FIG. 15. Bar graph showing toxicity of drug-free nanoparticle polyacrylate emulsions (left) and polymer films (right) against human dermal fibroblast cells.

FIG. 16. Bar graph showing antibacterial activity of drug-containing butyl acrylate-styrene films against S. aureus (849), MRSA (919), B. anthracis (848), and P. aeruginosa (10145). KG11-Ciprofloxacin methacrylamide emulsion. KG13-Ciprofloxacin acrylamide emulsion.

FIG. 17. Nicotine Standard Curve. The data is plotted in a spreadsheet as absorbency vs. concentration, where a trend line is added to obtain a linear equation (y=x−r), which is used to calculate unknown concentrations of nicotine in the release profile.

FIG. 18. Release profiles for encapsulated nicotine and nicotine added post-emulsion, with data reported as absorbance measured per time point. The 1% patches showed that the lower end of the range could be assessed accurately. The 1% encapsulated patch also showed a constant release pattern in respect to the 3% patch that had sharp increases in release through the various readings. A-3% non-encapsulated, B-3% encapsulated, C-1% non-encapsulated, D-1% encapsulated.

FIG. 19. Release profiles for encapsulated nicotine and nicotine added post-emulsion, with data reported as the cumulative amount of nicotine released at each time point. Even though the non-encapsulated patches releases nicotine at a higher rate initially, after 48 hours, the difference in the quantity of nicotine released is negligible. At 72 hours both the 1% and 3% patches release total amounts similar despite the nicotine being encapsulated or non-encapsulated. A-3% non-encapsulated, B-3% encapsulated, C-1% non-encapsulated, D-1% encapsulated.

FIG. 20. Extraction data from the emulsion patches were compared with extraction data from store brand patches, with data reported as amount released (mg) per time point. According to this extraction, the 7 mg and 21 mg store patch both release the same amount of nicotine per gram. A-7 mg commercial patch, B-21 mg commercial patch, C-3% encapsulated emulsion patch, D-1% encapsulated emulsion patch.

FIG. 21. Release profiles for encapsulated nicotine and store bought nicotine patches, with data reported as the cumulative amount of nicotine released at each time point. Again the 7 mg and 21 mg store patch show similar nicotine release characteristics. A-7 mg commercial patch, B-21 mg commercial patch, C-3% encapsulated emulsion patch, D-1% encapsulated emulsion patch.

FIG. 22. Release profiles for an entire patch size extracted for encapsulated nicotine and store bought nicotine patches, with data reported as the cumulative amount of nicotine released at each time point. A-7 mg commercial patch, B-1% non-encapsulated, C-1% encapsulated emulsion patch.

FIG. 23A-B. Inflammatory response (TNF alpha and IL-6 generation) to drug free poly(butyl acrylate-styrene) emulsion (NP0), or acrylated, penicillin-bound poly(butyl acrylate-styrene) emulsion (NP1), administered to a dermal abrasion at 9% solid content.

FIG. 24. Representation of the emulsion polymerization process with acrylated penicillin G monomer (NP1).

FIG. 25A-C. Cytotoxicity of saline (FIG. 25A) and drug free polyacrylate nanoparticle polymer films (FIGS. 25B and 25C) against human dermal fibroblast cells.

FIG. 26A-C. Treatment of a wound with drug-free polyacrylate nanoparticle emulsion. FIG. 26A: Excised tissue region on the back after 3 days of doctor-recommended treatment (polyacrylate not yet applied). FIG. 26B: Tissue after two days of polyacrylate emulsion application. FIG. 26C: Fully healed (10 days).

FIG. 27A-C. Treatment of a rope burn injury with drug-free polyacrylate nanoparticle emulsion. FIG. 27A: Three day old friction burn. FIG. 27B: Application of polyacrylate nanoparticle emulsion. FIG. 27C: 12 days post application.

FIG. 28A-B. Fully hydrated polyurethane sponges. Left sponge is coated with a drug-free polyacrylate nanoparticle emulsion. The right sponge was coated with high density polyurethane and caused deformation of the sponge when hydrated.

FIG. 29A-B. Day 2 after treatment of puncture wounds created during spider vein treatment using a drug-free polyacrylate nanoparticle emulsion. FIG. 29A: Site treated with emulsion. FIG. 29B: Site treated with petroleum-based emollient.

FIG. 30. The general reaction mechanism for the preparation of an acrylamide from an acyl chloride. In the reactions performed, R1-COCl is acryloyl chloride, and R₂—NH₂ refers any molecule with a primary amine group sterically available.

FIG. 31. Schematic of initial micelle formation during an emulsion polymerization, useful in producing compositions of the invention.

### DETAILED DESCRIPTION OF THE INVENTION

The present invention concerns biocompatible polymer materials (compositions) that exhibit mechanical and physical properties that are fundamental to many medical devices and treatment of many medical diseases and disorders. The compositions are composed of an emulsion of polymer and water, wherein the polymer comprises a copolymer of a base acrylate and a supporting monomer. Multiple applications of the compositions are contemplated. Thus, aspects of the invention include, but are not limited to, compositions comprising the emulsion, methods for preparing compositions of the invention, medical devices comprising the compositions, and methods of using the compositions by applying them to a desired site, e.g., a tissue, a surface of a medical device, or other substrate.

### EXEMPLIFIED EMBODIMENTS

1. A composition comprising an emulsion of polymer and water, wherein said polymer comprises a copolymer of a base acrylate and a supporting monomer.
2. The composition of claim 1, wherein said polymer is in the form of nanoparticles ranging from 10-400 nm.
3. The composition of embodiment 1, wherein said polymer is in a long chain format with nanoparticles intercrossing.
4. The composition of embodiment 1, wherein the composition is a medical device selected from a bandage, wound dressing, patch, implant, film, topical, injectable, ingestible, coating, interface, prosthetic, or adhesive.
5. The composition of any one of embodiments 1 to 4, wherein said base acrylate and said supporting monomer is present in a weight ratio range of 7:3 or 8:2 base acrylate to supporting monomer, and wherein said polymer comprise 1-30% of the emulsion.
6. The composition of any preceding embodiment, wherein said base acrylate is butyl acrylate, methyl acrylate or ethyl acrylate, and said supporting monomer is methyl methacrylate, methacrylate, styrene, methacrylamide, phenyl acrylate ethyl acrylate, or a combination of two or more of the foregoing.
7. The composition of embodiment 6, wherein said composition comprising two or more supporting monomers.
8. The composition of any preceding embodiment, further comprising at least one additive.
9. The composition of embodiment 8, wherein said additive comprises two or more additives.
10. The composition of embodiment 8, wherein said additive is covalently bound to said polymer.
11. The composition of embodiment 8, wherein said polymer is in the form of nanoparticles ranging from 30-150 nm, and wherein said additive is water insoluble and is incorporated into said nanoparticles.
12. The composition of embodiment 8, wherein said additive is a water soluble agent that is incorporated into the water phase of the emulsion during polymerization.
13. The composition of embodiment 8, wherein the additive is a water soluble agent that is incorporated into the water phase of the emulsion post-polymerization.
14. The composition of embodiment 8, wherein said additive is a bioactive agent that is covalently bound to said polymer, and wherein said additive is one or more additives selected from among polymyxin b, neomycin, bacitracin, prednisone, thiabendazole, lidocane, ciprofloxacin, penicillin G, penicillanic acid, cefaclor, mupirocin, amoxicillin, ampicillin, fusidic acid, clavulanic acid, dexamethasone, flucytosine, and nystatin.
15. The composition of embodiment 8, wherein said polymer is in the form of nanoparticles ranging from 10-400 nm, wherein said additive is a bioactive agent that is encapsulated within said nanoparticles, and wherein said additive is one or more additives selected from among tocopherols, aloe, nicotine, doxycycline, amphotericin B, clarithromycin, cefdinir, penicillin G, and penicillanic acid.
16. The composition of embodiment 8, wherein said additive is one or more natural preservatives and/or skin protectants selected from among ascorbic acid, citric acid, malic acid, glycerine, alkyl alcohols, lemongrass oil, limonene, cinnamon oil, lavender oil, tea tree oil, vitamin D, vitamin E, coconut oil, aloe vera, allantoin, cocoa butter, cod liver oil, citronellal oil, Eucalyptus oil, dimethicone, glycerin, hard fat, lanolin, mineral oil, petrolatum, white petrolatum, aluminum hydroxide gel, calamine, sodium bicarbonate, kaolin, zinc acetate, zinc carbonate, zinc oxide, and colloidal oatmeal.
17. The composition of embodiment 8, wherein said additive is a pH indicating dye, fluorescent dye, colored dye, or radioactive agent.
18. The composition of embodiment 8, wherein said additive is one or more thickening and/or hemostatic agents selected from among thrombin, potassium ferrate, carboxy methylcellulose, methyl cellulose, and citric acid.
19. The composition of embodiment 8, wherein said additive is a bittering agent.
20. The composition of embodiment 8, wherein said additive is an antimicrobial agent, antiviral agent, anticancer agent, pain reliever, analgesic, anti-inflammatory agent, or anesthetic agent.
21. The composition of embodiment 8, wherein said additive is a radioactive, fluorescent, or visualization (colored) agent.
22. The composition of embodiment 8, wherein said additive is a blood clotting agent.
23. The composition of embodiment 8, wherein said additive is a peptide, growth hormone, protein, blood component, plasma or combination of two or more of the foregoing.
24. The composition of embodiment 8, wherein said additive is a bioactive agent.
25. A method of preparing a composition, comprising:
combining a base acrylate and a supporting monomer with water, a surfactant, and a water soluble radical initiator to form a monomer suspension or emulsion; and polymerizing the monomer suspension or emulsion to form a polymer emulsion.
   26. The method of embodiment 25, wherein the weight percent of water in the monomer suspension or emulsion is 70 to 99%.
   27. The method of embodiment 25 or 26, wherein the weight ratio of base acrylate to supporting acrylate is in the range of 7:3 to 8:2.
   28. The method of any preceding embodiment, wherein the base acrylate is butyl acrylate, methyl acrylate or ethyl acrylate, and said supporting monomer is methyl methacrylate, methacrylate, styrene, methacrylamide, phenyl acrylate and/or ethyl acrylate.
   29. The method of any preceding embodiment, wherein the water soluble radical initiator is selected from the group consisting of peroxides, alkyl hydroperoxides, sodium salt of persulphate, ammonium salt of persulphate, potassium salt of persulphate, thiosulphates, metabisulphites, and hydrosulphides.
   30. The method of any preceding embodiment, wherein the surfactant is selected from the group consisting of lauryl alcohol, sodium dodecyl sulfate, lechitin, sodium lauryl sulfate, sodium dodecylbenzene sulphonate, sodium dioctyl sulphosuccinate, sodium or potassium salt of a fatty acid; sodium or potassium salt of a saturated fatty acid; and mixtures of any of the foregoing.
   31. The method of any one of embodiments 25 to 30, further comprising adding at least one additive to the polymer emulsion.
   32. The method of any one of embodiments 25 to 30, further comprising adding at least one additive to the monomer suspension or emulsion.
   33. The method of embodiment 31 or 32, wherein the additive is one or more selected from among polymyxin b, neomycin, bacitracin, prednisone, thiabendazole, lidocane, ciprofloxacin, cefaclor, mupirocin, amoxicillin, ampicillin, fusidic acid, clavulanic acid, dexamethasone, flucytosine, nystatin, tocopherols, aloe, nicotine, doxycycline, amphotericin B, clarithromycin, cefdinir, penicillin G, and penicillanic acid.
   34. The method of embodiment 31 or 32, wherein the additive is tone or more natural preservatives and/or skin protectants selected from among ascorbic acid, citric acid, malic acid, glycerine, alkyl alcohols, lemongrass oil, limonene, cinnamon oil, lavender oil, tea tree oil, vitamin D, vitamin E, coconut oil, aloe vera, allantoin, cocoa butter, cod liver oil, citronellal oil, Eucalyptus oil, dimethicone, glycerin, hard fat, lanolin, mineral oil, petrolatum, white petrolatum, aluminum hydroxide gel, calamine, sodium bicarbonate, kaolin, zinc acetate, zinc carbonate, zinc oxide, and colloidal oatmeal.
   35. The method of embodiment 31 or 32, wherein the additive is a pH indicating dye, fluorescent dye, colored dye, or radioactive agent.
   36. The method of embodiment 31 or 32, wherein the additive is a thickening and/or hemostatic agent selected from among thrombin, potassium ferrate, carboxy methylcellulose, methyl cellulose, and citric acid.
   37. The method of embodiment 31 or 32, wherein the additive is a bittering agent.
   38. The method of embodiment 31 or 32, wherein the additive is an antimicrobial agent, antiviral agent, anticancer agent, pain reliever, analgesic, anti-inflammatory agent, or anesthetic agent.
   39. The method of embodiment 31 or 32, wherein the additive is a radioactive, fluorescent, or visualization (colored) agent.
   40. The method of embodiment 31 or 32, wherein the additive is a blood clotting agent.
   41. The method of embodiment 31 or 32, wherein the additive is a peptide, growth hormone, protein, blood component, plasma or a combination of two or more of the foregoing.
   42. The method of embodiment 31 or 32, wherein at least two additives are added.
   43. The method of embodiment 32, wherein the additive comprises a polymerizable group.
   44. The method of embodiment 43, wherein the polymerizable group is an acrylate, acrylamide, or acrylamide functionality.
   45. The method of embodiment 26, further comprising deoxygenating the monomers, monomer suspension, or emulsion.
   46. A medical device comprising a dual applicator comprising a first chamber containing one or more enzymes to cleave an additive from the polymer; and a second chamber containing a composition of any one of embodiments 8 to 24.
   47. The device of embodiment 46, wherein the enzyme comprises a lipase and/or esterase.
   48. A method of protecting, promoting the healing or closure of, coagulating, covering, filling, and/or delivering an additive to, a tissue of a subject, comprising applying a composition of any one of embodiments 1 to 24 to hard or soft tissue.
   49. The method of embodiment 48, wherein the tissue is a wound.
   50. The method of embodiment 49, wherein the wound is an acute wound.
   51. The method of embodiment 49, wherein the wound is a chronic wound.
   52. The method of embodiment 49, wherein the wound is a cold sore.
   53. The method of embodiment 49, wherein the wound is a dermal abrasion.
   54. The method of embodiment 49, wherein the wound is a laceration.
   55. The method of embodiment 49, wherein the wound is a surgical incision.
   56. The method of embodiment 49, wherein the wound is a surgical excision.
   57. The method of any one of embodiments 48 to 56, wherein the composition is applied intra-operatively.
   58. The method of embodiment 57, wherein the composition is applied intra-operatively to a site of Dura leakage or vascular leakage.
   59. The method of embodiment 48, wherein the tissue is intact skin or a mucous membrane.
   60. The method of embodiment 48, wherein the composition is applied to adhere and repair injuries to soft tissue.
   61. The method of embodiment 48, wherein the composition is applied to adhere and repair injuries to hard tissue.
   62. The method of embodiment 48, wherein the tissue is bone.
   63. The method of embodiment 48, wherein the tissue contains elastin.
   64. The method of embodiment 48, wherein the tissue is intact skin.
   65. The method of embodiment 48, wherein the tissue is the retina.
   66. The method of embodiment 48, wherein the tissue is intact skin, and the composition comprises nicotine.
   67. The method of embodiment 48, wherein the composition is applied as a medical sealant.
   68. The method of embodiment 67, wherein the composition is applied to a partially implanted medical device.
   69. The method of embodiment 68, wherein the partially implanted medical device is a bone screw, bone pin, or stent.
   70. The method of embodiment 49, wherein the composition is applied as a medical adhesive.
   71. The method of embodiment 49, wherein the composition is applied as a hemostatic agent.
   72. The method of embodiment 49, wherein the composition is applied as a permanent filler subcutaneously.
   73. The method of embodiment 49, wherein the composition is applied to an absorbent material.
   74. The method of embodiment 73, wherein the absorbent material is a polyurethane, cotton, polyvinyl alcohol, or synthetic absorptive fiber.
   75. The method of embodiment 73, wherein the absorbent material is a sponge, foam, gauze, bandage, pad, or wound dressing.
   76. A method of coating a medical device, comprising applying a composition of any one of embodiments 1 to 24 to a surface of the medical device.
   77. The method of embodiment 76, wherein the composition is applied to a biodegradable implantable medical device.
   78. The method of embodiment 76, wherein the composition is applied to a permanent implantable medical device.
   79. The method of embodiment 76, wherein the composition is applied to a removable implantable medical device.
   80. The method of embodiment 76, wherein the composition provides a biocompatible interface between a medical device and a biological tissue.
   81. The method of embodiment 80, wherein the medical device is a medical electronic device.
   82. The method of embodiment 80, wherein the medical device is a prosthesis.

As used herein, the term “applying”, in the context of compositions of the invention, means contacting the composition on, in, and/or around a desired anatomical site, such as a wound or an unwounded site on or in the body. The compositions of the invention can be applied to any intact or wounded, hard or soft tissue of the body (e.g., connective, muscle, nervous epithelial, or combination of two or more types). The composition is kept in contact with the anatomical site to achieve a desired result, such as one or a combination of covering and/or protecting the site, promoting wound healing, closure, or sealing of tissue, inducing or promoting coagulation, filling a void, or delivering an agent (e.g., a bioactive agent) such as a drug or biologic compound, etc.

As used herein, the term “subject” includes humans and non-human animals.

Following are examples that illustrate procedures for practicing the invention. These examples should not be construed as limiting. All percentages are by weight and all solvent mixture proportions are by volume unless otherwise noted.

### Example 1

### Drug-free Polymeric Nanoparticle Emulsions

Drug free polymeric nanoparticle emulsions were made according to Table 1.

TABLE 1
Formulation of emulsion polymerizations containing butyl acrylate (BA) with
either styrene (Sty) or methyl methacrylate (MMA) co-monomers.

| | | BA:Co- | | | Max | Young's | Particle |
|---|---|---|---|---|---|---|---|
| | Co- | monomer | Surfactant | Initiator | Stress | Modulus | Size |
| Emulsion | Monomer | ratio | (%) | (%) | (MPa) | (MPa) | (nm) |
| CNP5 | Sty | 7:3 | 3 | 0.5 | 1.165 | 0.269 | 46.0 |
| CNP6 | MMA | 8:2 | 3 | 0.5 | 0.449 | 0.365 | 76.9 |
| CNP7 | Sty | 8:2 | 3 | 0.5 | 0.146 | 0.102 | 35.9 |
| CNP9 | Sty | 7:3 | 5 | 1 | 0.643 | 0.615 | 53.8 |
| CNP10 | Sty | 8:2 | 5 | 1 | 0.594 | 0.423 | 51.3 |
| CNP12 | MMA | 7:3 | 3 | 0.5 | 1.671 | 0.771 | 68.7 |
| CNP13 | MMA | 7:3 | 5 | 0.5 | 0.984 | 0.647 | 61.4 |
| CNP14 | MMA | 8:2 | 5 | 1 | 0.296 | 0.326 | 62.3 |
| CNP15 | Sty | 7:3 | 1 | 1 | 0.605 | 0.451 | NA |
| CNP16 | Sty | 7:3 | 1 | 0.5 | 0.675 | 0.458 | 91.5 |
| CNP17 | MMA | 7:3 | 1 | 0.5 | 1.105 | 0.478 | 89.2 |

Additionally, homopolymers of MA (methacrylate), MMA (methyl methacrylate), and ethyl acrylate (EA), as well as copolymers of EA:MA, EA:MMA, EA:Sty, and MA:MMA, were made. The referred ratio of the monomers is 7:3, using 1-3% surfactant.

### Methods of Preparation

### Polymeric Nanoparticle Emulsion Preparation

A polymeric nanoparticle emulsion consists of two types of acrylates: a) base acrylate monomer, composed of butyl acrylate, methyl acrylate, or ethyl acrylate plus b) a supporting acrylate monomer, composed of methyl methacrylate, methacrylate, styrene, phenyl acrylate, methacrylamide, or ethyl acrylate in a ratio of 8:2 or 7:3 base acrylate to supporting acrylate, with the exact monomers tailored to the specific application need. The acrylates compose the solid content of the emulsion and will be approximately 1-30% (20% preferred) (w/w) of the total solution. Water will be 70-99% of the total solution, and may contain salts and/or buffers. To create the micelles in the solution, 1-5% surfactant, in this case of dodecyl sulfate, will be used. The 1-5% will be based out of the total solids in the emulsion, in this case, the acrylates used. A radical initiator at 0.5-1.5% (w/w) will be used to start the polymerization. Without limitation, as an example the emulsion may be made in the following general fashion. First, the acrylates are measured according to the total volume of emulsion prepared and mixed together in an oxygen free flask. This is then heated to 70-90° Celsius. Once heated, surfactant is added and mixed by mechanical means. Water and surfactant are then added and the system is purged of all oxygen. The resulting solution is mixed, the radical initiator is added, and the system is purged of oxygen again. The resulting solution is left to mix until complete polymerization is achieved.

For example to make 50 g of polyacrylate emulsion:
Acrylate Monomer Phase- to make 30% solids
   Weigh 10.5 g of either base acrylate into a vessel
   Add 4.5 g of supporting acrylate
   Flush the vessel with either nitrogen or argon.
   Heat the vessel to 70-90° C. for 10 minutes with gentle stirring
Add 150 mg of surfactant to the acrylate monomer phase, continue stirring, maintain temperature at 70-90° C.
Add 40 g water to the acrylate monomer phase (alternatively the SDS can be dissolved in the water and then added together to the acrylate monomer phase)
Continue stirring 15-30 minutes
Add 75 mg radical initiator
Mix 6 to 8 hours maintaining 70-90° C. temperature
Resulting emulsion can be further diluted with water, buffer, saline, or polar solvents such as alcohols.

### Example 2

### Covalently Bound Nanoparticle Drug Formulations

Covalently bound nanoparticle drug formulations were made according to the general procedure below:
Water soluble or insoluble drugs, small molecules or bioactive compounds may be covalently bound to the polyacrylate nanoparticles by adding the compound of interest together with the base acrylates, prior to micelle formation.
   For example to make 50 g of polyacrylate emulsion:
Acrylate Monomer Phase- to make 30% solids
   Weigh 10.5 g of either base acrylate into a vessel
   Add 4.5 g of supporting acrylate
Add desired bioactive agent, up to 20% of the solid content weight
Flush the vessel with either nitrogen or argon
Heat the vessel to 70-90° C. for 10-30 minutes with gentle stirring
Add 150 mg of surfactant, continue stirring, maintain temperature at 70-90° C.
Add 40 g water
Continue stirring 15-30 minutes
Add 75 mg radical initiator
Mix 6 to 8 hours

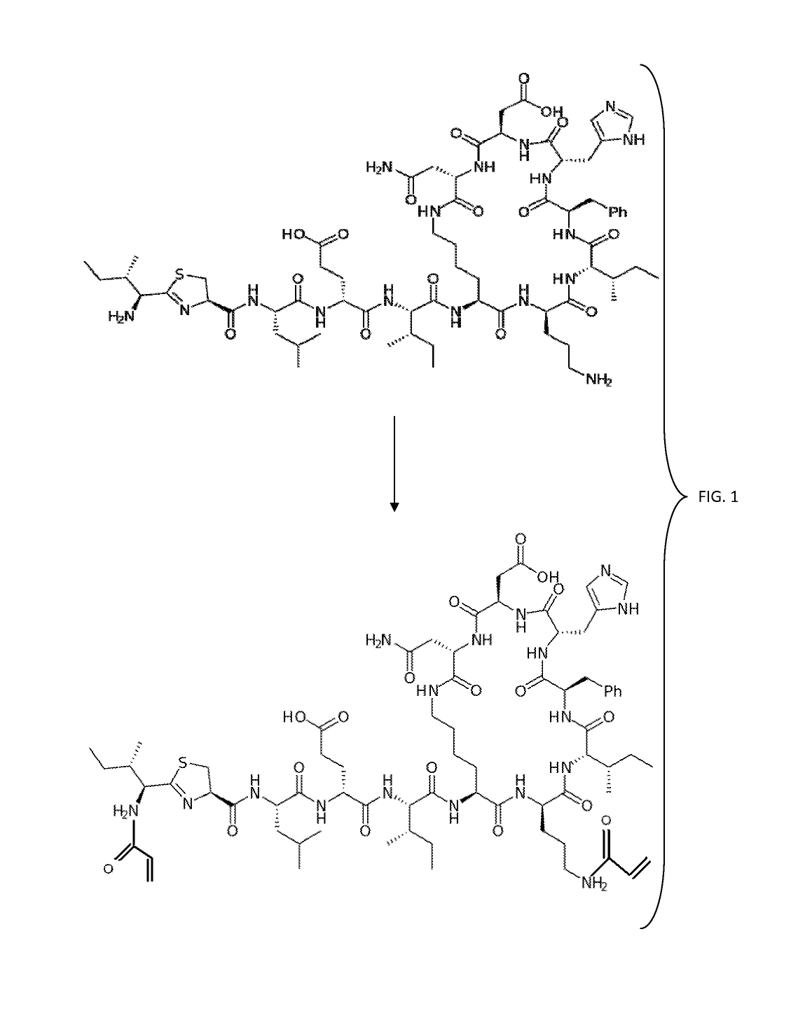

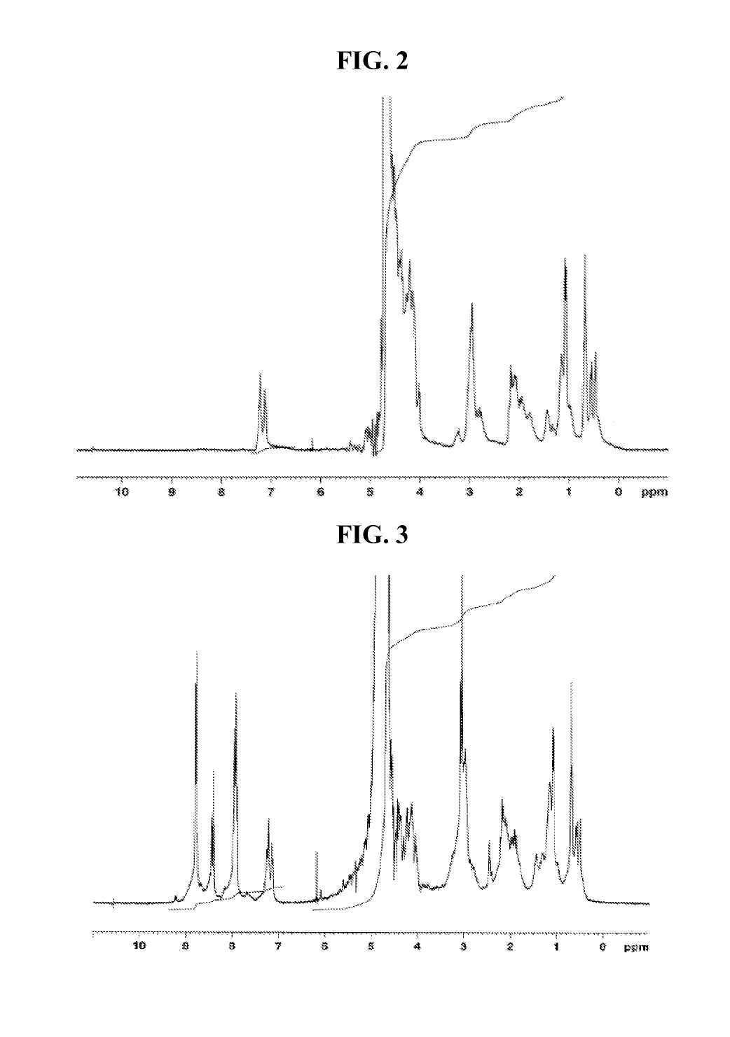

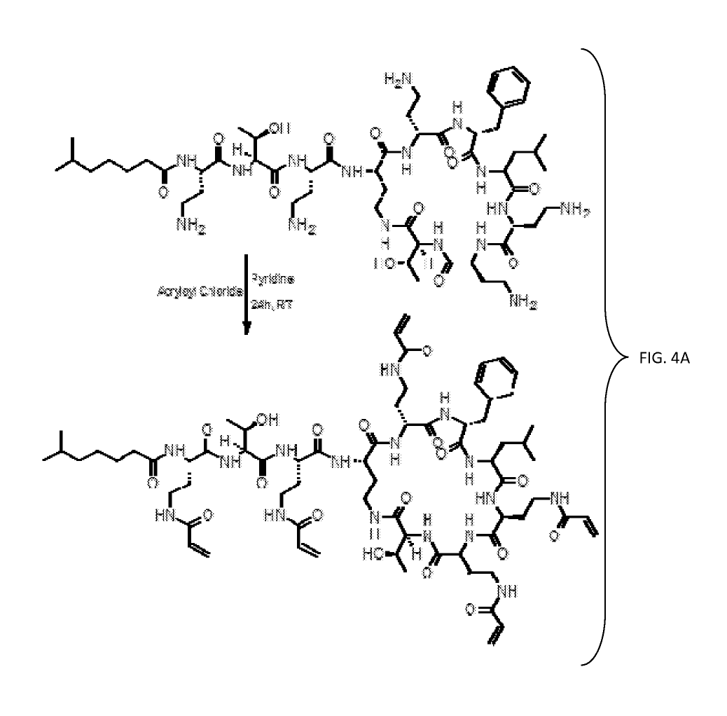

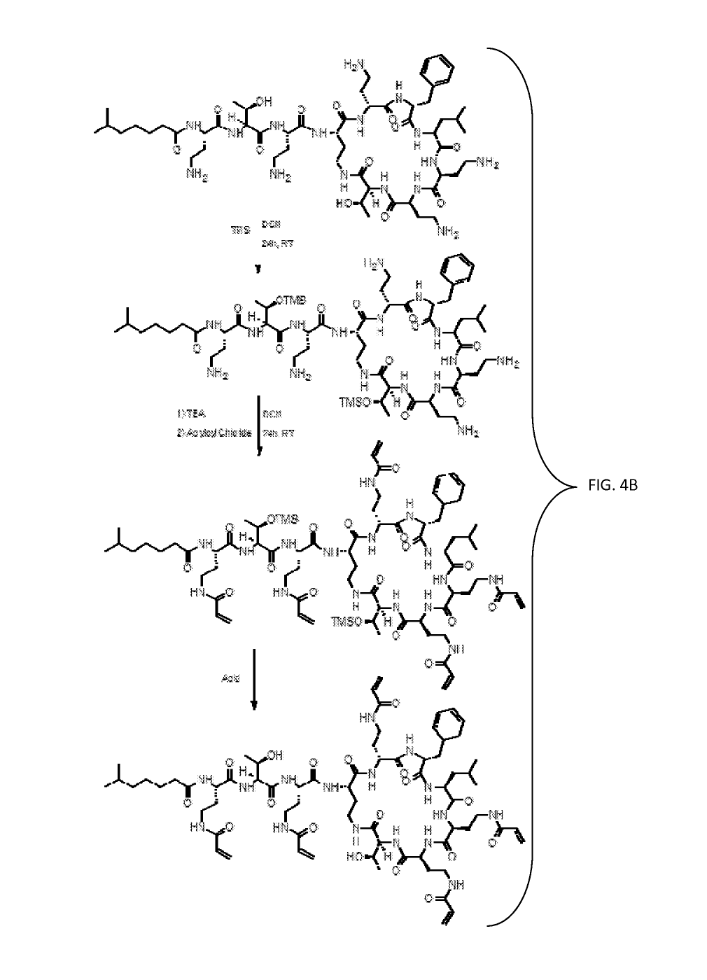

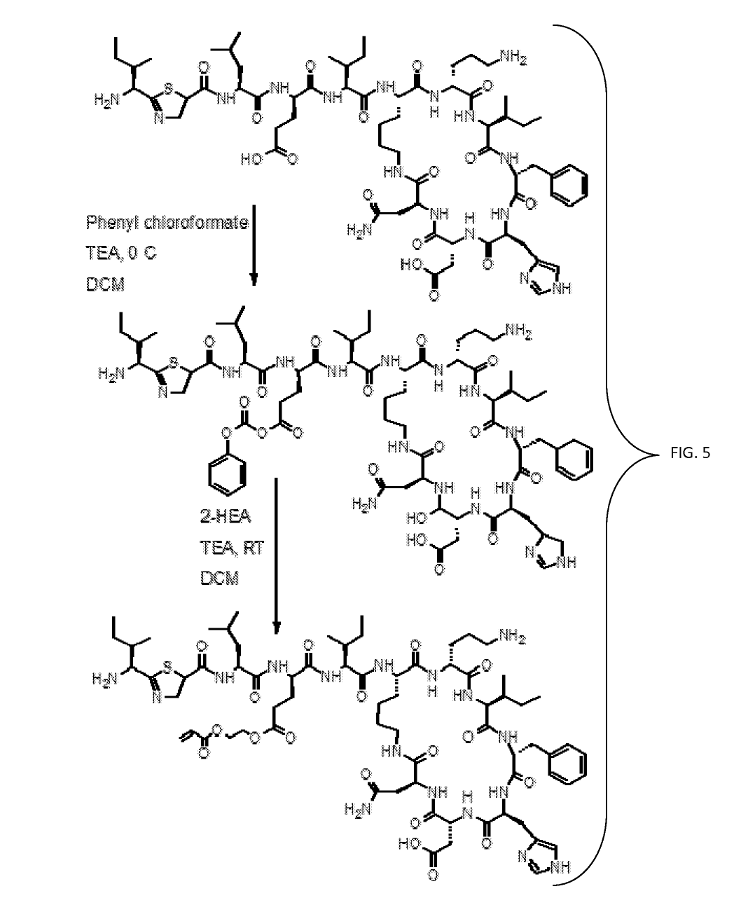

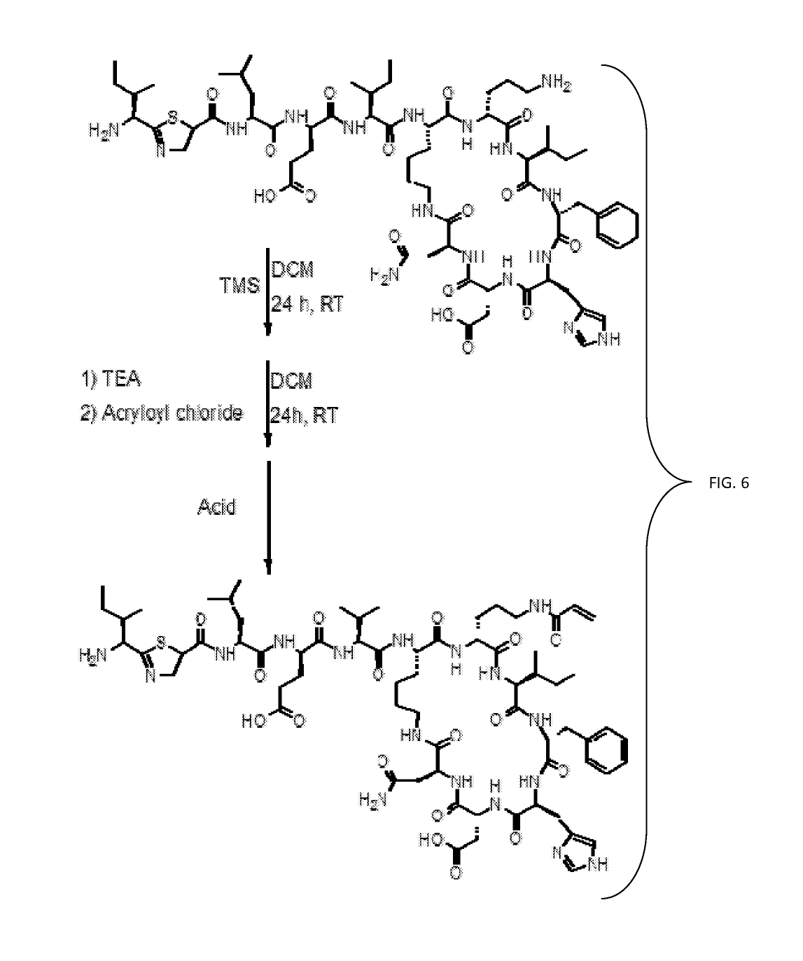

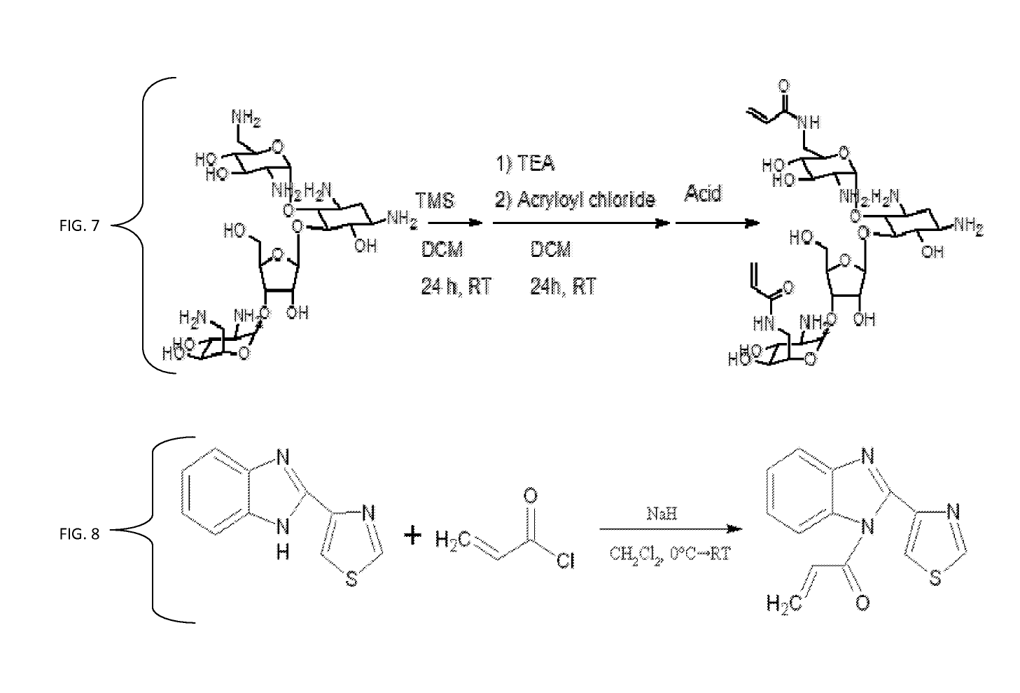

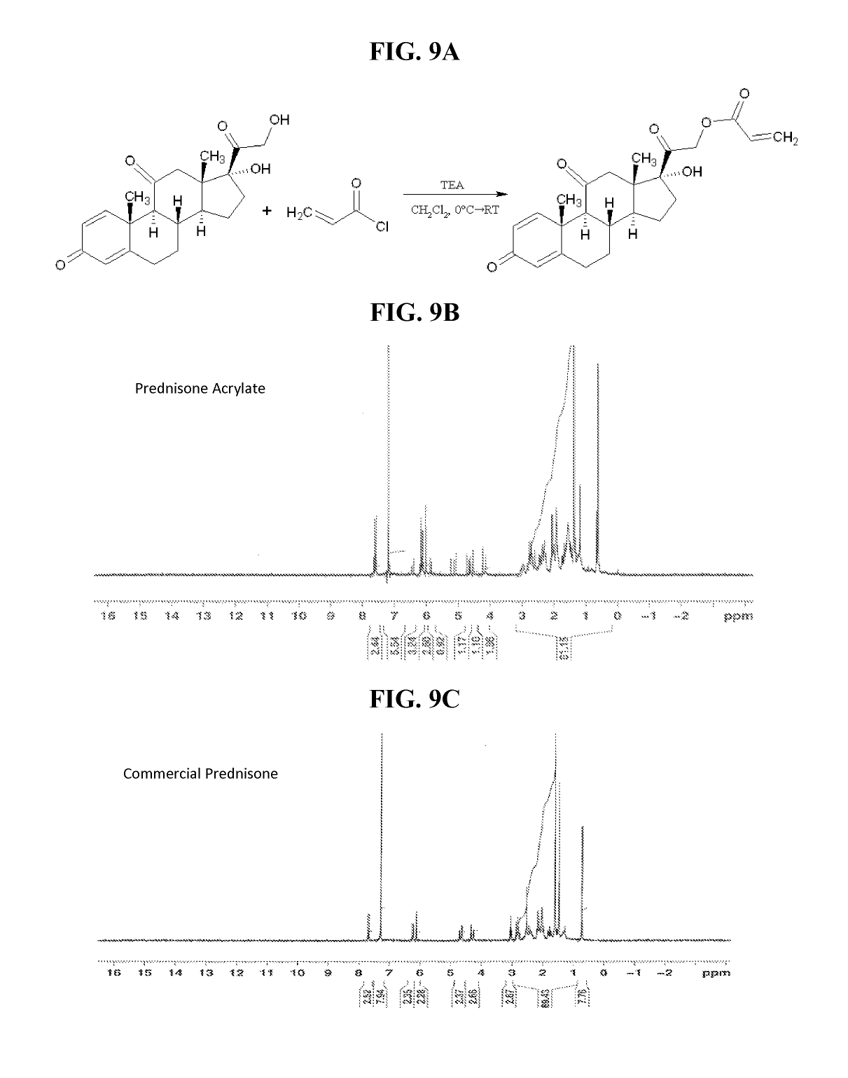

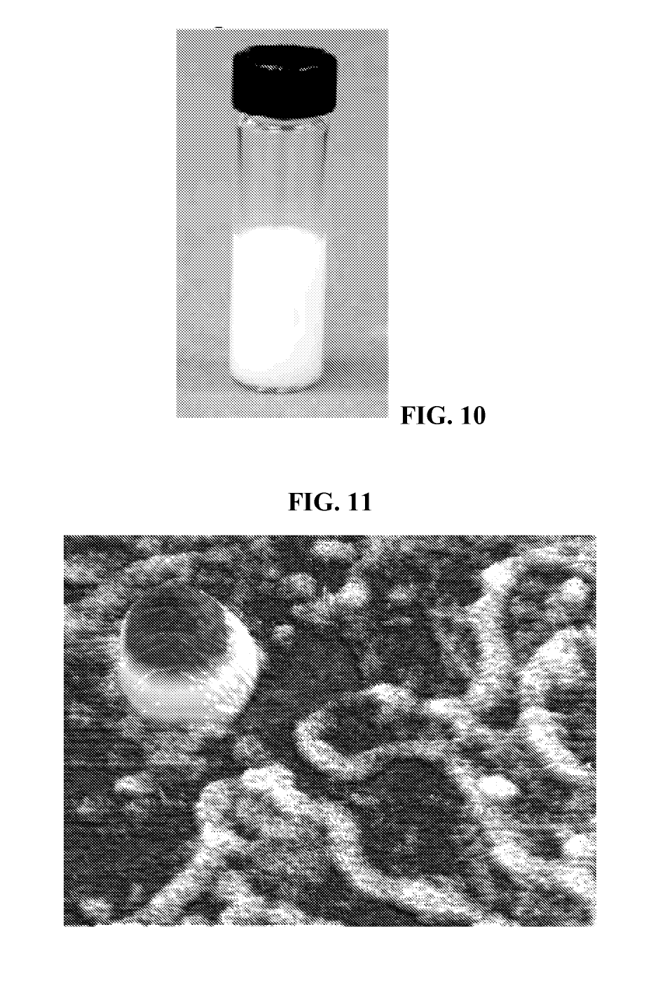

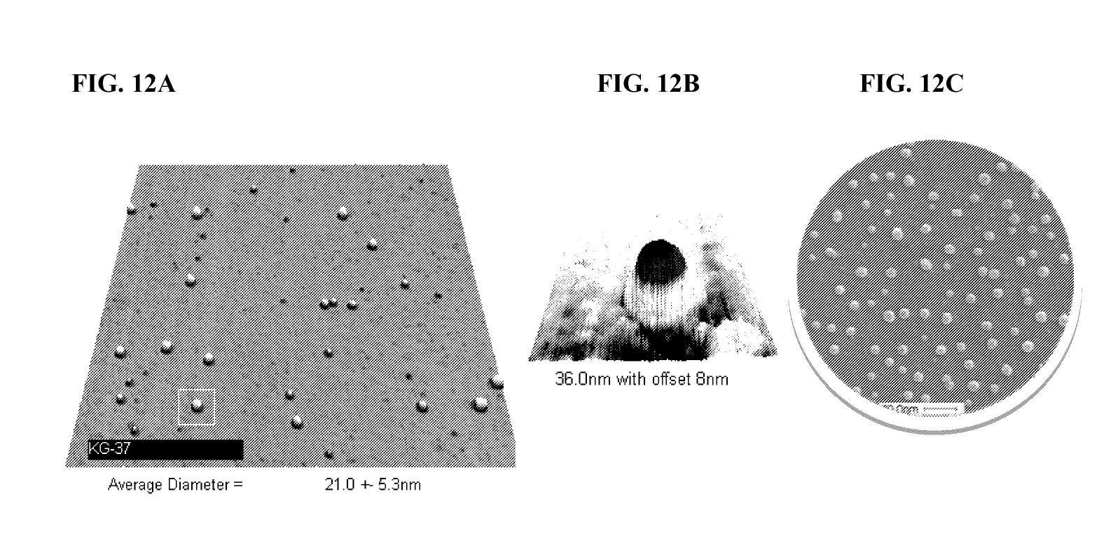

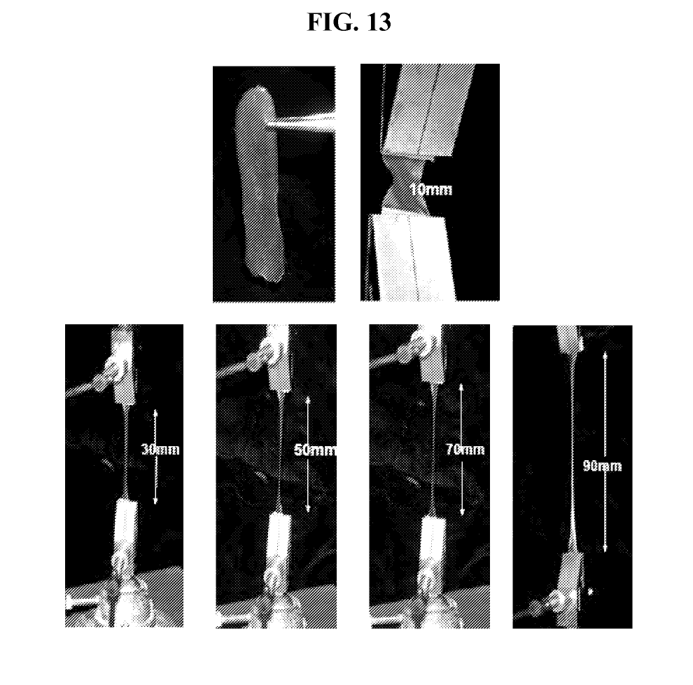

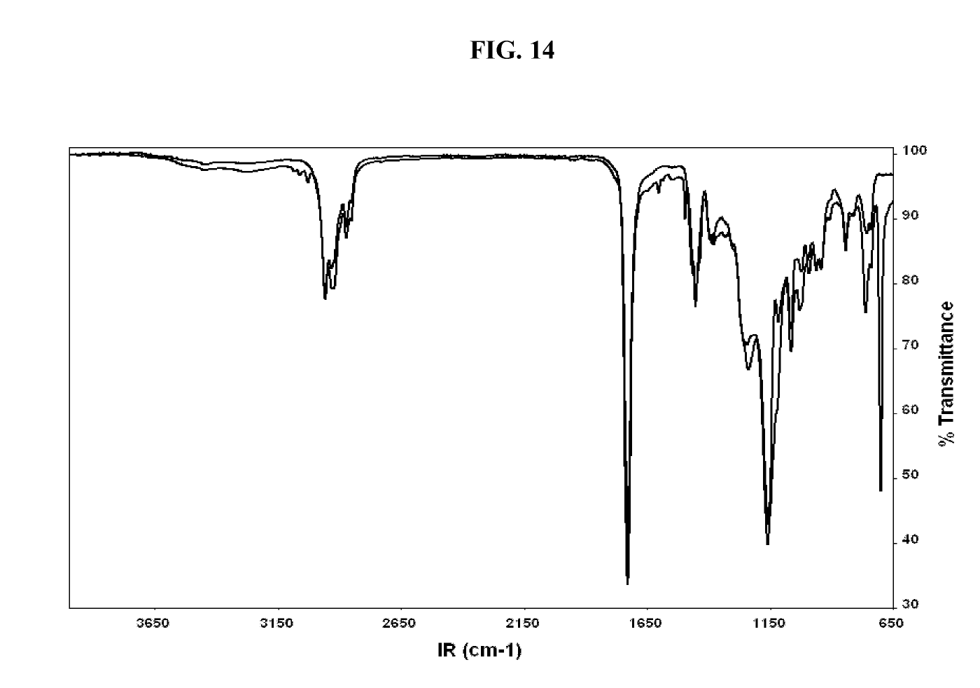

Without limitation, examples of compounds that can be modified to permit covalent binging in the nanoparticles include polymyxin B, bacitracin, ciprofloxacin, prednisone, lidocaine, penicillin, neomycin, ampicillin, amoxicillin, ceflacor, fusidic acid, clavulanic acid, dexamethasone, hydrocortisone, flucytosine, nystatin, aspirin, mupriocin, thiabendazole, erythromycin, amphoteracin, clarithromycin, doxycycline, nicotine, tocopherols, aloe-emodin. FIG. 1 illustrates the general reaction scheme for the preparation of drug-bound nanoparticles. FIGS. 2-10 illustrate several examples of drug bound nanoparticles of the present invention.

### Acrylation of a Primary or Secondary Amine or Alcohol Groups.

Modification will follow the same process as has been described in the publications of PI Greenhalgh and Turos using acryloyl chloride as the acrylating agent and targeting the primary and secondary amine or alcohol sites for acrylation using a mild amine base.³⁻⁷ Here, we will use Bacitracin as a model as shown in FIG. 2. Bacitracin is modified through the following acrylation process, workup and purification via column chromatography to afford an acrylamide analogue of the parent drug molecule. Variations in the procedure are likely to occur for each of the drug molecules described and covered in the described technology due to the difference in solubilities among the different additive molecules, however, all will follow the same foundation of for acrylate/acrylamide formation. Step 1—Protect any free interfering groups with an appropriate protecting group and conditions conducive to the specific additive molecule. Examples include use of trimethylsillyl chloride, ethyl chloroformate, and acetone for protection of carboxylic acid moieties. Step 2—Activate the free amine or alcohol group using an amine base or other activation method for the alcohol, such as anhydride formation, followed by addition of acryloyl chloride. Reaction should be kept at room temperature for no longer than 24 hours to prevent self-polymerization.

### Acrylation of an Amide Group

Acrylation of amide groups on bioactive molecules serves as a slightly more challenging acrylation, but the end result is an imide that is more easily cleaved and therefore serves a unique purpose and provides distinct release profiles from acrylate and acrylamide additives. For acrylation here, a stronger base is employed in order to deprotonate the amide, typically sodium hydride. The remaining process follows suite with that of the amine and alcohols, acrylation using a form of acryloyl chloride, followed by acid workup and column purification. Examples here include acrylation of penicillin G.

### Acrylation at a Carboxylic Acid Site of the Additive:

Acrylation at the carboxylic acid site is permitted through a modified route using 2-hydroxyethyl acrylate (2-HEA) in lieu of acryloyl chloride.^{3,5,6} This modification provides a longer linkage to the polymer, providing more visibility outside of the nanoparticles as well as easier access to the bound molecule for enzymatic cleavage. In this procedure, the carboxylic acid group is activated by conversion to an anhydride moiety under basic conditions, followed by acrylation with 2-HEA under base.

### Drug Delivery:

Covalently bound drugs can also be used for drug delivery through intact skin by using a dual applicator with one chamber containing enzymes to cleave the drug, the other containing the nanoparticle with bound drug. Appropriate enzymes include lipases and esterases. Lipases will cleave acrylates and acrylamides. Esterases will cleave acrylates. In wounded skin, the enzymes would be naturally produced by the host, or by bacterial, fungal or cancer cells.

### Additives:

Polymer modifying acrylate additives can also be incorporated into the polymer by adding the additive to the base acrylate: co-monomer phase. Categories include surfactants to stabilize emulsion polymers, chain transfer agents and other polymerization modifiers to control molecular weight, plasticizers to increase flexibility, stabilizers to prevent polymer degradation, and crosslinkers used to modify polymer networks. Up to 10% of the acrylate monomer phase may consist of additives, drugs, etc (10% of the “solids”).

### Example 3

### Nanoparticles with Encapsulated Drug and Other Drug Containing Nanoparticle Formulations

Nanoparticle encapsulated water insoluble compound drug formulations were made according to the general procedure described herein. A polymeric nanoparticle emulsion can be prepared form a single monomer, but preferably include at least two types of acrylates such as those pairings listed in Table 1 and Example 1. For example, a) base acrylate monomer, composed of butyl acrylate, methyl acrylate or ethyl acrylate plus b) a supporting acrylate monomer, composed of methyl methacrylate, methacrylate or styrene, in a ratio of 8:2 or 7:3 base acrylate to supporting acrylate, with the exact monomers tailored to the specific application need. The acrylates compose the solid content of the emulsion and will be approximately 1-30% (w/w) of the total solution. Water will be 70-99% of the total solution. To create the micelles in the solution, 1-5% surfactant, in this case of dodecyl sulfate, will be used. The 1-5% will be based out of the total solids in the emulsion, in this case, the acrylates used, and will vary depending on the need of the additive encapsulated. A radical initiator at 0.5-1.5% (w/w) will be used to start the polymerization. The steps to make the proper emulsion are the following. Without limitation, as an example, the emulsion may be made in the following general fashion. First, the acrylates are measured according to the total volume of emulsion prepared and mixed together in an oxygen free flask. This is then heated to 70-90° Celsius. Once heated, surfactant is added and mixed by mechanical means. Water and surfactant are then added and the system is purged of all oxygen. The resulting solution is mixed, the radical initiator is added, and the system is purged of oxygen again. The resulting solution is left to mix until complete polymerization is achieved.

For example, to make 50 g of nicotine encapsulated polyacrylate emulsion:
Weigh 10.5 g of either base acrylate into a vessel
Add 4.5 g of supporting acrylate
Add 200 mg of nicotine
Flush the vessel with either nitrogen or argon.
Heat the vessel to 70-90° C. for 10 minutes with gentle stirring
Add 150 mg of surfactant, continue stirring, maintain temperature at 70-90° C.
Add 40 g water
Continue stirring 15-30 minutes
Add 75 mg radical initiator
Mix 6 to 8 hours

A specific amount of emulsion (from 1 ml to 4 ml) can then be applied on an inert or dermal surface for it to air-dry. On a dermal surface, the nicotine can be absorbed and thus function as a transdermal system delivery. A release profile can be done when a film is formed on an inert surface and then added to phosphate buffer and incubated at 36° Celcius. At different time periods, from 1 hr-72 hrs, the PBS is collected and measured on a spectrophotometer within the 256-320 nm ranges. The concentration of nicotine can be determined using the equation derived from the nicotine standard curve. The standard curve is prepared by mixing a known concentration amount of nicotine into PBS and then performing 10-15 serial dilutions to determine the concentration of nicotine at different dilutions.

Without limitation, examples of compounds that can be modified to permit covalent attachment to the nanoparticles include:

### Erythromycin

### Amphotericin B

### Clarithromycin

### Cefdinir

### Doxycycline Monohydrate

### Penicillin G

Penicillanic acid

### Nicotine

### Tocopherols

### Aloe

### Drug Delivery:

Encapsulated drugs can be used for drug delivery through either a wound or intact skin/tissue. The polymer itself will not penetrate intact skin, but the encapsulated drug can be released from the particle to migrate through the skin, depending upon that drug's properties as well as other additives included in the microemulsion formulation to enhance such delivery, including both water soluble and water insoluble excipients. An example of an encapsulated drug that could penetrate intact skin is nicotine.

Non-encapsulated, unbound drug emulsions can also be generated using the general method described herein, wherein a water soluble drug of interest is added to the final emulsion (after the 6-8 hour final mixing step).

### Example 4

### Release Profiles of Drug Containing Nanoparticles

The drug-bound nanoparticles have demonstrated release profiles making them suitable for drug delivery. There are two methods for examining the release profile of nicotine from transdermal patches. The first method involves measuring a piece of the nicotine patch weighting at about 2-3 g and placing it in a container with 45-50 ml of phosphate buffer solution. The system is then incubated at 36-37° Celsius for 3 days, the duration of the experiment. Within those 3 days, at intervals of 1 hr, 2 hr, 4 hr, 8 hr, 12 hr, 24 hr, 48 hr and 72 hr 5 ml of the PBS solution in the system is collected to be analyzed in a spectrophotometer where the reading at 260 nm will be used to determine the concentration at that particular interval. The second method involves a smaller portion of the patch at about 0.7-1.5 g. The nicotine patch is placed in container with 5 ml of PBS and incubated at 36-37° C. for 3 days. Utilizing the same intervals as in the first method, the 5 ml of PBS is collected for spectrophotometric analysis and replaced with 5 ml of fresh PBS.

In order to determine the concentrations of the collected samples in the nicotine release profiles, a nicotine standard curve is prepared. The standard curve is made by placing a known concentration of nicotine in 4 ml of PBS and then serially diluting 15 times. These 15 samples are then measured at 260 nm in a UV/VIS spectrophotometer to obtain the absorbency at each dilution. The data is then plotted as absorbency vs. concentration, where a trendline is added to obtain a linear equation (y=mx+b), which is used to determine the amount of nicotine released from the polymer.

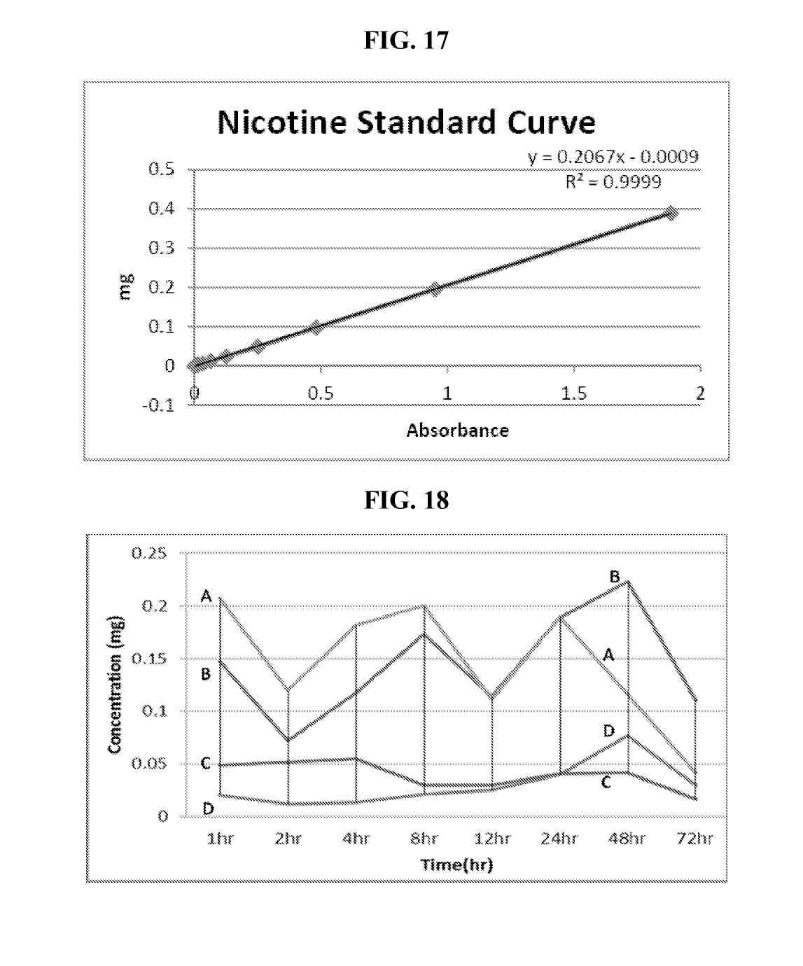

The system used to measure the amount of nicotine in solution is based upon the standard curve seen in FIG. 17. Absorbance was measured and the amount of nicotine was calculated using this graph.

Nicotine was incorporated within the emulsion either through encapsulation or by adding to the system post-polymerization. A nicotine patch, made by drying the polymer emulsion and was compared against a commercially available nicotine patch. Each patch was prepared to weigh 2-3 g and was placed in a container with 5 ml of phosphate buffer solution. The system was then incubated at 36-37° Celsius for 3 days. Within those 3 days, at intervals of 1 hr, 2 hr, 4 hr, 8 hr, 12 hr, 24 hr, 48 hr and 72 hr the 5 ml of the PBS is removed and the amount of nicotine is measured using a UV/VIS spectrophotometer at 260 nm wavelength. At each time point, 5 mL of fresh PBS replaces the 5 mL taken out for spectrophotometer measurements.

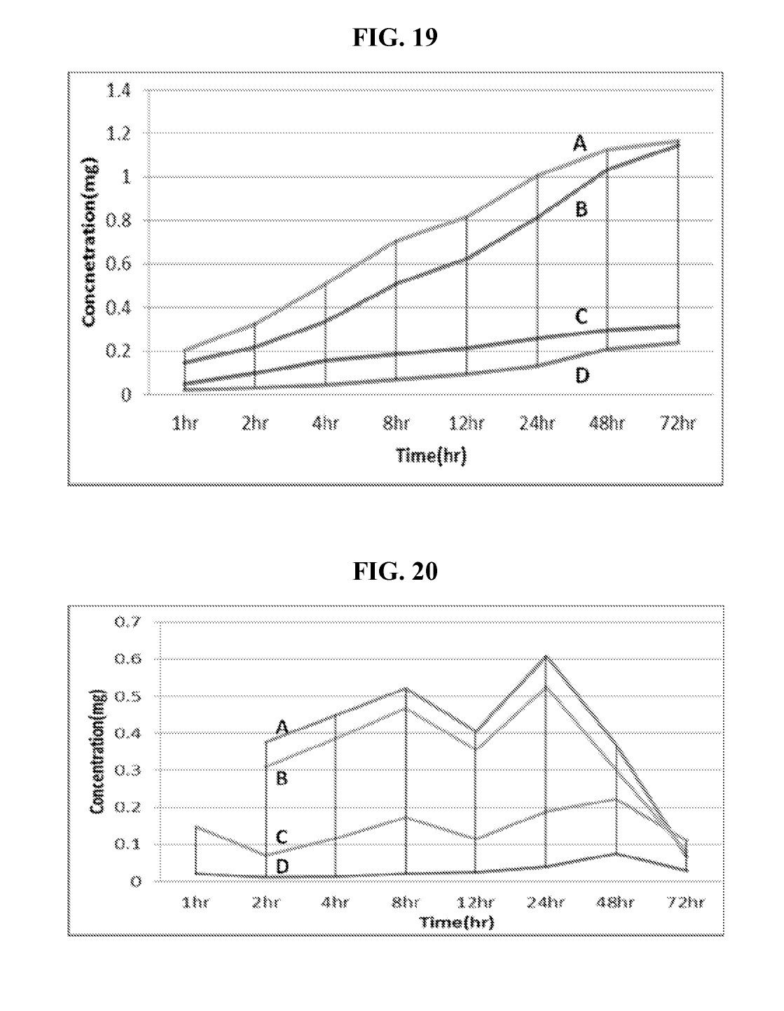

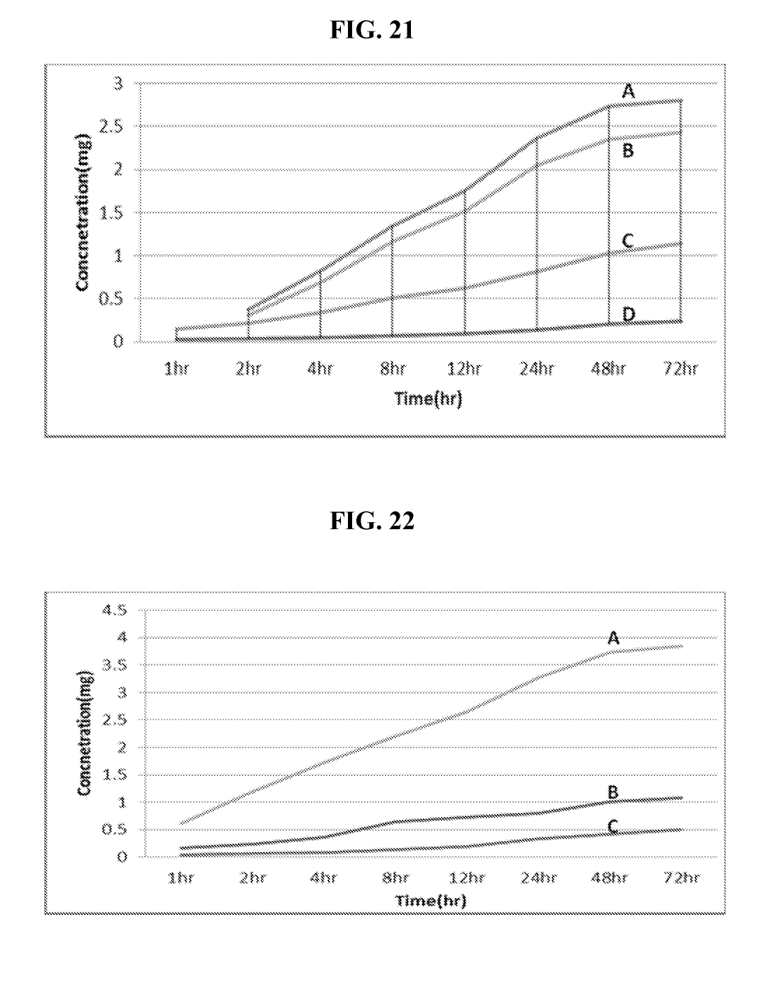

The graphs are shown in FIGS. 17-22.

The release profile was also investigated using the entire patch instead of a 2-3 g sample, and 10 mL of PBS was used instead of 5 mL. Since the only difference between the 7 mg and 21 mg store patch was the size and not nicotine concentration, only the 7 mg patch was used for the extraction experiment. The system was then incubated at 36-37° Celsius for 3 days like the previous with the same measurement intervals of 1 hr, 2 hr, 4 hr, 8 hr, 12 hr, 24 hr, 48 hr and 72 hr. The 10 mL of the PBS is removed and the amount of nicotine is measured using a UV/VIS spectrophotometer at 260 nm wavelength. At each time point, 10 mL of fresh PBS is used to replace the 10 mL taken out for measurements.

Based upon the data collected, the encapsulated patch has a more consistent release profile compared to the non-encapsulated nicotine patch. This ensures a more controlled delivery and lower risk of a drug overdose or over exposure, if this is a concern. If there were a need for a high initial burst, then the non-encapsulated patch would be preferential.

The adaptability of the polymer system used allows stringent control over the release and drug absorption, tailoring the release profile to each specific application desired, and control over how much or how little drug in released at a time.

### Example 5

### Cytotoxicity and Inflammation

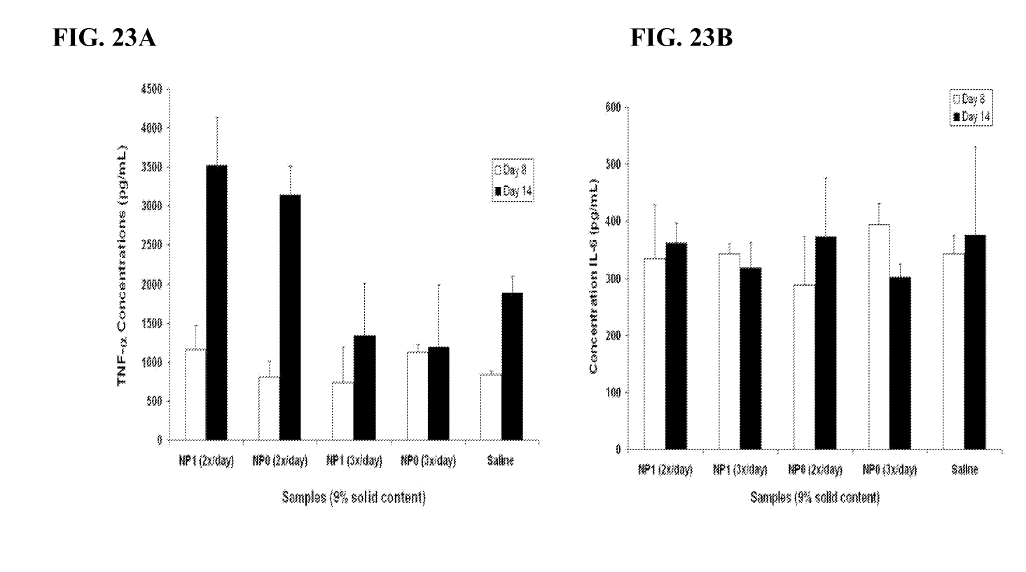

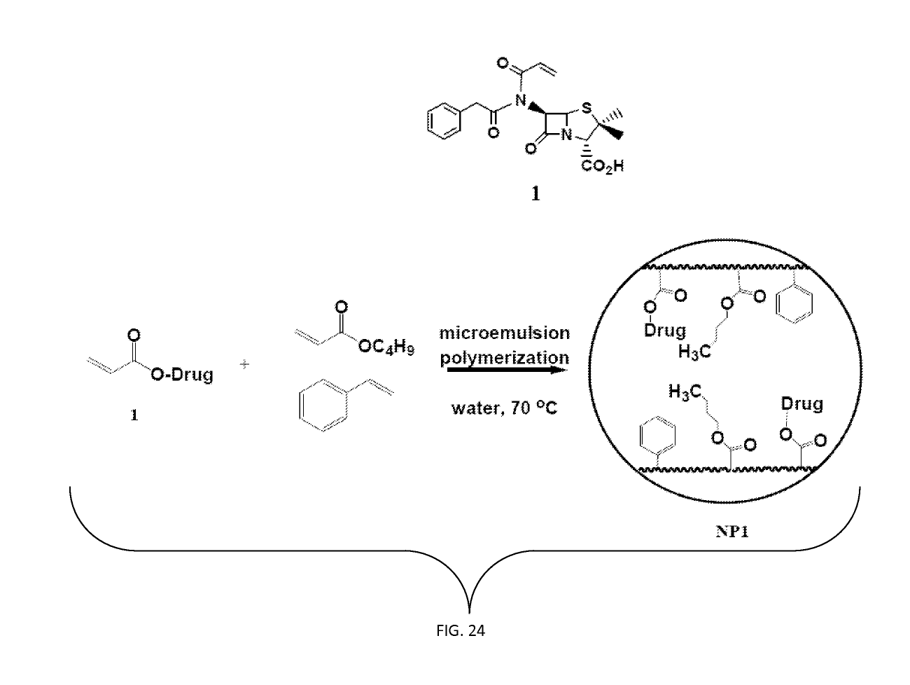

Two polyacrylate nanoparticle emulsion preparations were used, drug free and penicillin g bound. The polymerization process for penicillin G is shown in FIG. 24. Mice were subjected to a wound model via tape stripping as previously reported in Greenhalgh and Turos, 2009. FIGS. 23A-B show no significant inflammatory response (TNF alpha content of the blood serum from wounded and treated mice) when the emulsions were applied to mice for 7 days.

Additionally, mice with a dermal abrasion were treated topically two times a day with poly(butyl acrylate-styrene) nanoparticle emulsion with acrylated penicillin drug monomers incorporated into the polymer, at 9% solid content (0.1 mL/application). The abrasion was fully healed by day 5 and fur re-growth was fully established by day 14 of the study.

In comparison, mice with a dermal abrasion treated with saline solution three times a day showed obvious inflammation. At day 3 there was indication of a possible bacterial infection. Wound healing was setback an additional 2 days, and was still not fully healed by day 8.

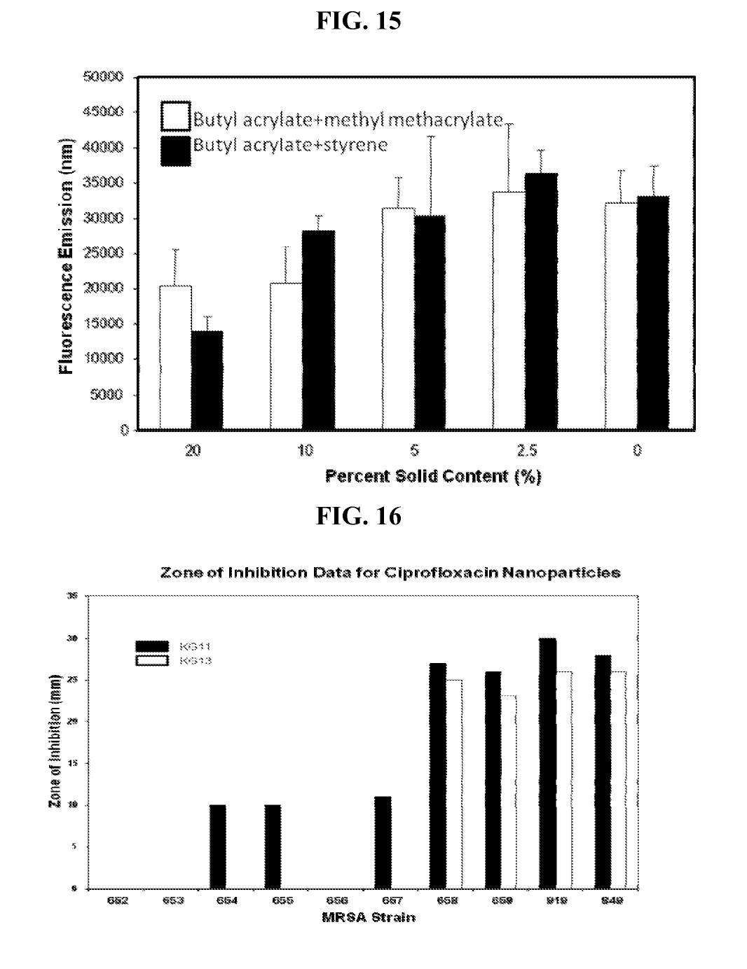

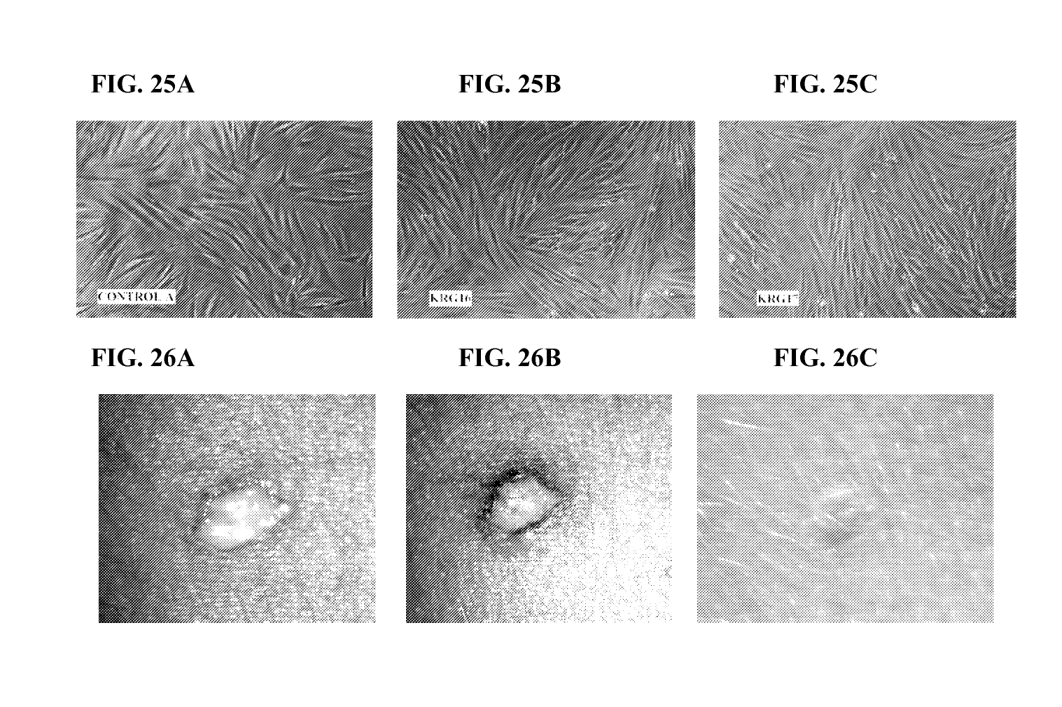

FIGS. 15 and 25A-C illustrate the growth of normal human dermal fibroblast cells in the presence of drug free polyacrylate nanoparticle emulsions, demonstrating a lack of cytotoxicity

### Example 6

### Antibacterial Activity of Ciprofloxacin-Bound Nanoparticles

FIG. 16 shows the antibacterial activity of ciprofloxacin-bound poly(butyl acrylate-styrene) emulsions against common pathogens found in topical and internal wounds. S. aureus (849), MRSA (919), B. anthracis (848), and P. aeruginosa (10145). KG11-Ciprofloxacin methacrylamide emulsion. KG13-Ciprofloxacin acrylamide emulsion.

### Example 7

### Hemostatic Activity

The polymeric emulsion, with and without additives incorporated, stops bleeding on contact, has a fast set up time, and forms a protective film to prevent infection. This occurs through a charge attraction between the blood components and the overall negative charge of the nanoparticles due to specific choice of the surfactant, in this case sodium dodecyl sulfate. The result of this interaction is immediate precipitation of the polymer with the blood, with the solid precipitate forming a protective film over the bleeding wound to prevent further blood loss. When a drop of the composition is placed on a bleeding wound, moisture is sucked into the wound bed. The polymer has a negative charge, which interacts with the positive charge of the blood component, and causes coagulation. The bleeding is stopped because a film is formed with the blood and polymer. This seals the exit point in the wound, perforation, hemorrhage, or incision site. Additionally, this features works with any biological fluid containing positively charged components. Film formation also occurs in situ, forming a solid polymer at the site of administration to seal, coat, or plug surgical areas. In order to enable the composition to expedite blood coagulation, it may be applied as needed until bleeding ceases. For example, the hemostatic abilities of the emulsion were tested in a puncture wound by administering the drug free polyacrylate nanoparticle emulsion of the current invention. Additionally, the drug free polyacrylate nanoparticle emulsion was administered to a minor bleeding laceration. Finally, the drug free polyacrylate nanoparticle emulsion was administered to an arterial laceration in a canine hind paw to stop bleeding. In all cases, the emulsion stopped the bleeding.

### Example 8

### Surface Coating

The emulsion has been applied to a number of materials, both porous and non-porous, with the intent of providing a non-degradable surface coating. Applications include both medical and non-medical uses. The properties of the film are tailored by adjusting the acrylate monomers and ratios to fit the need of the application. Coatings act as an anti-biofouling surface, a compatible biological interface, or as an active delivery vehicle. Additionally, the coatings' and/or films' elasticity will permit mirrored physical properties to elastic soft tissues in the body, including tissues comprising the skin, lungs, heart, uterus, diaphragm, and vasculature. The elasticity also makes the polymer applicable to absorbent medical devices such as foams, sponges, gauze, grafts, and other wound dressings and bandages that require expansion to function properly when applied. The film is capable of formation on any material, including but not limited to glass, Teflon, metals, polyurethane, cotton, polyvinyl alcohol, synthetic materials and other polymers and medical grade materials. The polymer coating can be thickened by applying multiple times and heat set to create multiple layers. Additional applications will seamlessly bind together with no evidence of lamination. FIGS. 28A-B illustrate sponges coated with the nanoparticle emulsions of the current invention. Polyurethane sponges were coated with the polyacrylate nanoparticle emulsion of the present invention then hydrated using sterile saline. This was compared with commercially available sponge coated with high density polyurethane as is the industry standard for a water-proof and antimicrobial barrier for foam based wound dressings. With each sponge, the coating created a water proof barrier. While a commercial product deformed the sponge, the emulsion of the current invention did not and allowed equal coverage and expansion of the foam, which would translate into complete coverage of a wound.

### Example 9

### Surgical Excision of Tissue for Biopsy

Excision of a growth on the lower dorsal torso of a female subject for diagnostic biopsy. Recommended treatment protocol was the use of Vaseline and bandages to cover wound and reduce scar tissue formation, which was carried out for the first 3 days post excision. Patient had dermal sensitivity to both Vaseline and the adhesive present in the bandage, causing severe irritation of excision site and surrounding tissue. Excision produced exudate as a result of the wound care protocol.

The wound was cleaned with hydrogen peroxide, the polyacrylate emulsion was applied using the ball rod applicator (approx. 0.1 mL dosage) once a day for the first 3 days, then every other day until Day 8. No further application was necessary past this point. Complete wound granulation was observed as early as Day 10 with no evidence of contraction and no instance of scar tissue formation.

Exudates were drastically reduced by Day 2 along with redness and irritation with the polyacrylate emulsion treatment. Wound bed granulation was visible throughout the process. No instance of wound bed contraction was observed. Evidence points to a highly favorable cosmetic outcome. FIGS. 26A-C illustrate treatment of a wound with drug free polyacrylate nanoparticle emulsion. Left: Excised tissue after 3 days of doctor-recommended treatment. Middle: Tissue after two days of emulsion application. Right: Fully healed (10 days).

### Example 10

### Friction Burn Wound

Patient received a contact friction burn from a thin rope during routine exercise. Patient cleaned wound with peroxide. The wound was not covered with bandages or antimicrobials. The wound on the anterior surface of the lower limb began to form eschar on Day 3 post-injury. The wound on the posterior surface of the lower limb was in a high friction and no granulation or eschar formation was observed by Day 4. Both sites were treated with product on Day 6 post-injury, where more of the product was applied to the posterior wound that remained open, sensitive to air and contact, and was slightly exudating. Treatment with the product continued for 7 days on the posterior wound, and for 9 days for the anterior wound.

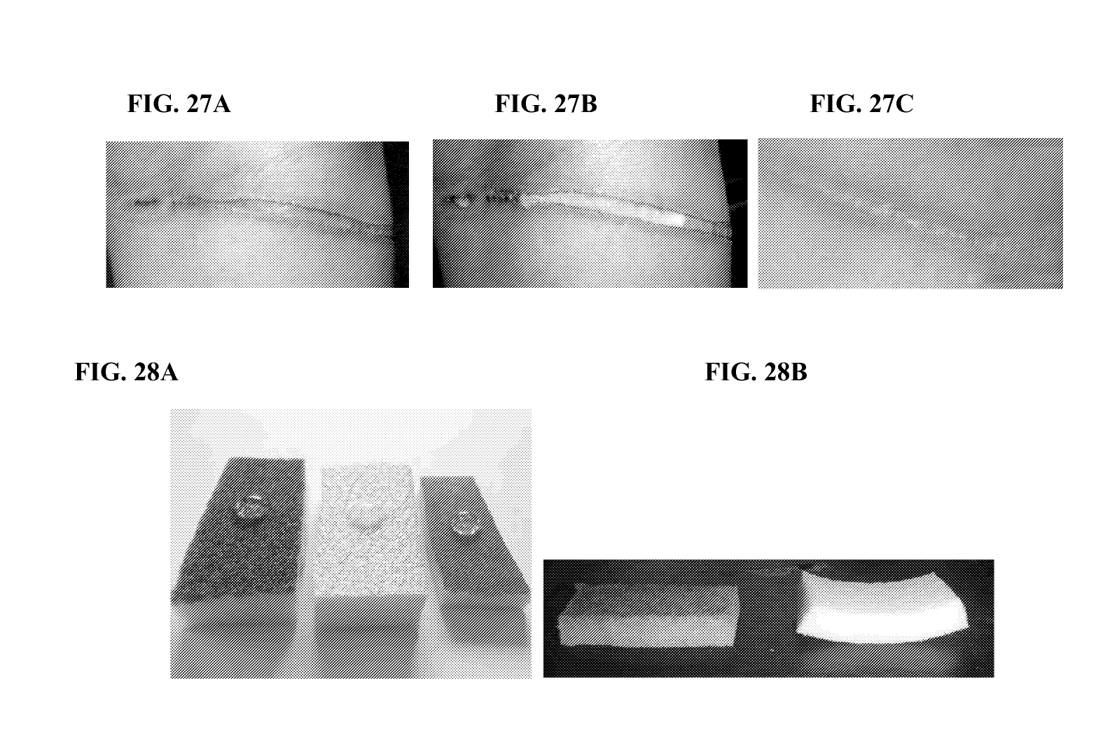

Treatment on the exposed dermal burn wound with the hydrogel emulsion provided immediate, complete coverage of the wound and reduced sensitization of the wound to air and friction from clothing and skin contact. The posterior wound resolved itself within 7 days, with no evidence of contraction or scar tissue formation. The anterior wound maintained eschar until Day 9, and contracture remains evident on Day 12 (right aspect of wound in above image). FIGS. 27A-C illustrate treatment of a wound with drug free polyacrylate nanoparticle emulsion: Three day old friction burn. Middle: Application of polyacrylate nanoparticle emulsion. Right: 12 days post application.

### Example 11

### Canine Arterial Laceration

A canine experienced a deep laceration to the plantar surface of the hindpaw between the metacarpal and carpal pads. Wound produced arterial blood spray that could not be subdued by compression or bandage. Application of a large quantity (approx. 1 mL) of the polyacrylate to the wound caused the bleeding to immediately cease and was successfully transported to an animal hospital for surgery. Veterinarian observed a laceration to the arterial network in the hindpaw and surgically repaired the artery followed by closure of the skin using non-resorbable suture. The hind paw was wrapped in gauze to prevent the dog from pulling the stitches. Subsequently, the emulsion was continuously applied over the outer layer of stitches every day and recover with the gauze. On Day 8 the dog pulled the outer stitches and required a secondary visit to the veterinarian for re-suturing. The composition was applied to stop bleeding when the wound was re-opened and continued to be applied after the second set of sutures was in place. No additional bandages were used after the second surgery. The incision site was fully healed within the next 10-14 days with no instance of infection and no visible scar formation.

### Example 12

### De-Gloving of Canine Forelimb

Canine presented with severe de-gloving injury of the lower hind limb (over 70% of the affected area) as well as a distal tibia fracture and other internal injuries. The wound was treated with OroGen T(rf) product (a solution containing active canine-originating growth factors) and covered with a Tefla pad for the first 10 days, then covered with a Scilon polymer dressing and Tefla dressing for the next 9 days as the composition was not yet available. The Scilon dressing was removed on day 19 and the area was covered with the emulsion. Due to the amount of hydration in the tissue bed, the film remained tacky and the veterinarian applied petroleum impregnated gauze over the area followed by a cotton/gauze bandage after 10 minutes set time. After 3 days the bandage was changed and slight bleeding was observed. The wound was re-covered with the emulsion. After 10 days, the film was observed saturated with exudate and was gently removed with a sponge. No tissue ingrowth into the composition was observed, allowing minimal issues during film removal, a significant improvement over typical Tefla and Scilon dressing changes. Treatment remains in progress with epithelialization improving daily.

### Example 13

### Sealant for Use with Partially Inserted Medical Devices

For use with a stent or catheter, apply the composition every 2-3 days. The composition can be applied by using a spray or a brush-on applicator. The stent, screws, or other inserts are inserted into the body and the polymer is applied to seal the gap between the insert and the tissue. Histological evidence suggests tissue will not grow into the film, therefore, application will ensure that no tissue is damaged when the stent or insert is removed. The polymer bandage itself peels off easily from solid intact skin with no additional damage caused during removal.

### Example 14

### Spider Vein Treatment

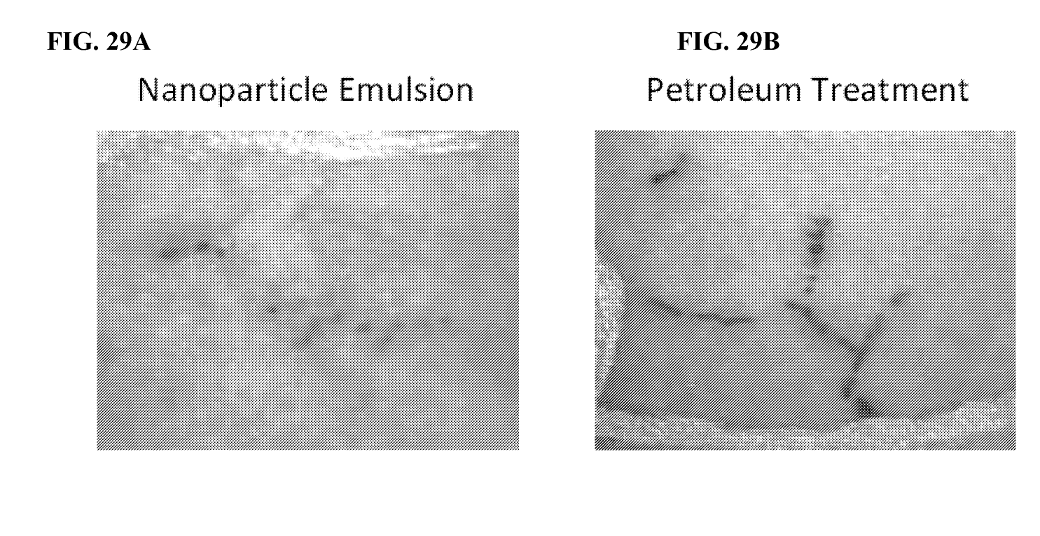

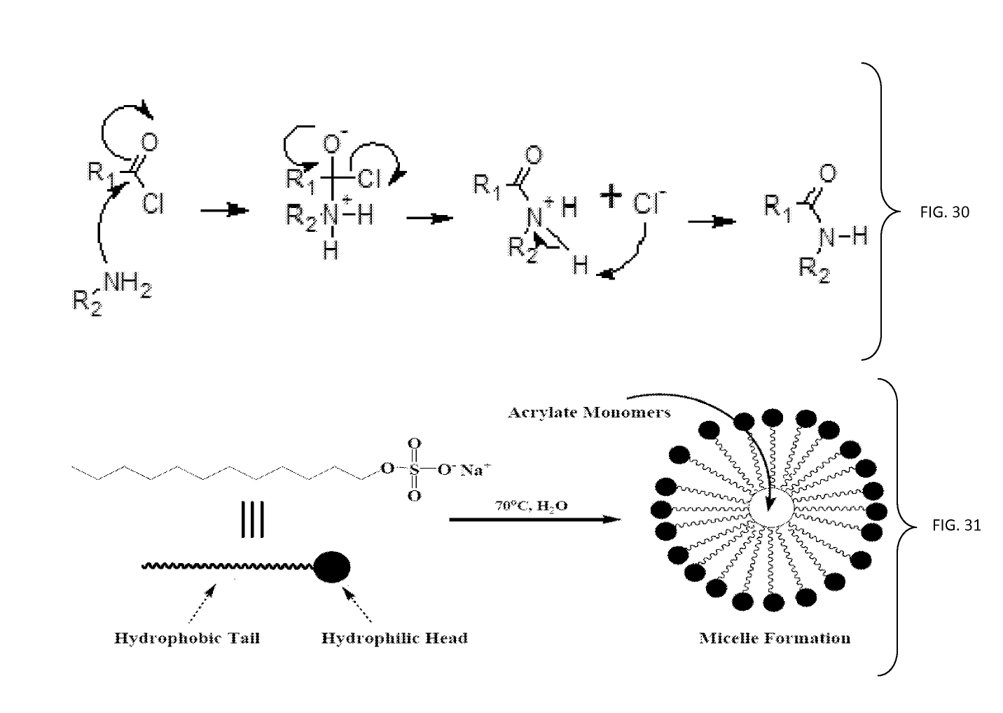

The emulsion is applied to minor puncture wounds created during cosmetic spider vein treatments, thereby preventing scab formation when applied immediately upon injury. Complete healing observed within 2 weeks, compared to petroleum product requiring 4 weeks for healing along with scab formation. Minimal scar formation observed with emulsion treatment. FIGS. 29A-B illustrate treatment of wounds created during spider vein treatment using drug-free polyacrylate nanoparticle emulsion.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Benita, S. 1999. Prevention of topical and ocular oxidative stress by positively charged submicron emulsion. Biomed. Pharm. 53:193-206
2. Cerrada, M. L., J. L. de la Fuente, M. Fernandez-Garcia, E. L. Madruga. 2001. Viscoelastic and mechanical properties of poly(butyl acrylate-styrene) copolymers. Polymer 42:4647-4655.
3. Chang, F.-Y.; J. E. Peacock Jr., D. M. Musher, P. Triplett, B. B. MacDonald, J. M. Mylotte, A. O'Donnell, M. M. Wagener, and V. L. Yu. 2003. Staphylococcus aureus bacteremia: Recurrence and the impact of antibiotic treatment in a prospective multicenter study. Medicine 82:333-337.
4. Cleary, J. D. 1996. Amphotericin B formulated in a liquid emulsion. Ann. Pharmacother. 30:409-412.
5. Costerton, J. W., P. S. Stewart, and E. P. Greenberg. 1999. Bacterial biofilms: A common cause of persistent infections. Science 284:1318-1322.
6. Danishevskii, S. L., B. Kalinin. 1965. Toxicological characteristics of synthetic polymeric materials. Itogi Nauki, Farmakologiya, Toksikologiya 1965:123-159.
7. De, T. K., E. J. Bergey, S. J. Chung, D. J. Rodman, D. J. Bharali, P. N. Prasad. 2004. Polycarboxylic acid nanoparticles for ophthalmic drug delivery: an ex vivo evaluation with human cornea. J. Microencapsulation 21:841-855.
8. Deegan, R. J. 1992. Propofol: a review of the pharmacology and applications of an intravenous anesthetic agent. Am. J. Med. Sci. 304:45-49.
9. Doenicke, A. W., M. F. Roizen, J. Rau, W. Kellermann, J. Babl. 1996. Reducing pain during propofol injection: the role of the solvent. Anesth. Analg. 82:472-474.
10. Goto, Y., K. Hiramatsu, and M. Nasu. 1999. Improved efficacy with non-simultaneous administration of netilmicin and minocycline against methicillin-resistant Staphylococcus aureus in in vitro and in vivo models. Intl. J. Antimicrob. Agents. 11:39-46.
11. Gu, H., P. L. Ho, E. Tong, L. Wang, B. Xu. 2003. Presenting vancomycin on nanoparticles to enhance antimicrobial activity. Nano Lett. 3:1261-1263.
12. Guignard, B., J. M. Entenza, P. Moreillon. 2005. b-lactams against methicillin-resistant Staphylococcus aureus. Current Opin. Pharma. 5:479-489.
13. Hicks, R., A. K. Satti, G. D. H. Leach, I. L. Naylor. 1989. Characterization of toxicity involving hemorrhage and cardiovascular failure, caused by parenteral administration of a soluble polyacrylate in the rat. J. Appl. Tox. 9:191-198.
14. Hussain, F., M. Hojjati. 2006. Review article: Polymer-matrix nanocomposites, processing, manufacturing, and application: An Overview. J. Comp. Materials 40:1511-1565.
15. Junginger, H. E., J. C. Verhoef. 1998. Macromolecules as safe penetration enhancers for hydrophilic drugs-a fiction? Pharm. Sci. Tech. Today 1:370-376.
16. Kirsh, R., R. Goldstein, J. Tarloff, D. Parris, J. Hook, P. Bugelski. 1988. An emulsion formulation of amphotericin B improves the therapeutic index when treating systemic murine candidiasis. J. Infect. Dis. 158:1065-1070.
17. Kugelberg, E., T. Norstrom, T. K. Petersen, T. Duvold, D. I. Andersson, and D. Hughes. 2005. Establishment of a superficial skin infection model in mice by using Staphylococcus aureus and Streptococcus pyogenes. Antimicrob. Agents Chemother. 49:3435-3451.
18. Lamb, K. A., C. Washington, S. S. Davis, S. P. Denyer. 1991. Toxicity of amphotericin B emulsion to cultured canine kidney cell monolayers. J. Pharm. Pharmacol. 43:522-524.
19. Lance, M. R., C. Washington, S. S. Davis. 1995. Structure and toxicity of amphotericin B/triglyceride emulsion formulations. J. Antimicrob. Chemother. 36:119-128.
20. Levy, M. Y., S. Benita. 1989. Design and characterization of submicronized o/w emulsion of diazepam for parenteral use. Int. J. Pharm. 54:103-112.
21. Liliete C. Souza, L. C., R. C. Maranhao, S. Schreier, A. Campa. 1993. In-vitro and in-vivo studies of the decrease of amphotericin B toxicity upon association with a triglyceride-rich emulsion. J. Antimicrob. Chemother. 32:123-132.
22. McCormick, J. K., J. M. Yarwood, and P. M. Schlievert. 2001. Toxic shock syndrome and bacterial superantigens: An Update. Annu Rev. Microbiol. 55:77-104.
23. McGrath, J. J., L. Purkiss, M. Eberle, W. R. McGrath. 1994. Long-term effects of a cross-linked polyacrylate superabsorbent in the hamster. J. Appl. Tox. 15:69-73.
24. Mcgrath, J. J., L. Purkiss, M. Christian, N. H. Proctor, W. R. Mcgrath. 1993. Teratology study of a cross-linked polyacrylate superabsorbent polymer. J. Am. College Tox. 12:127-137.
25. Mizushima, Y. 1996. Lipid microspheres (lipid emulsions) as a drug carrier—an overview. Adv. Drug. Deliv. Rev. 20:113-115.
26. Morones, J. R., J. L. Elechiguerra, A. Camacho, K. Holt, J. B. Kouri, J. T. Ramirez, M. J. Yacaman. 2005. The bactericidal effect of silver nanoparticles. Nanotechnology 16:2346-2353.
27. Ne, A., T. Xia, L. Madler, N. Li. 2006. Toxic potential of materials at the nanolevel. Science 311:622-627.
28. Nolen, G. A., A. Monroe, C. D. Hassall, J. Iavicoli, R. A. Jamieson, G. P. Daston. 1989. Studies of the developmental toxicity of polycarboxylate dispersing agents. Drug Chem. Tox. 12:95-110.
29. Norrby, S, N., C. E. Nord, and R. Finch. 2005. Lack of development of new antimicrobial drugs: a potential serious threat to public health. Lancet. Infect. Dis. 5:115-119.
30. Peters, W. J., R. W. Jackson, D. C. Smith. 1974. Studies of the stability and toxicity of zinc polyacrylate (polycarboxylate) cements (PAZ). J. Biomed. Mater. Res. 8:53-60.
31. Peters, W. J., R. W. Jackson, K. Iwano, D. C. Smith. 1972. Biological response to zinc polyacrylate cement. Clin. Ortho. Rel. Res. 88:228-233.
32. Reich, A., A. Kaplan, R. Gisler. 1996. Thermosetting polyacrylate powder coating compositions. Eur. Pat. Appl. 9 pp.
33. Renjis, T., V. Tom, P. Suryanarayanan, G. Reddy, S. Baskaran, T. Pradeep. 2004. Ciprofloxacin-protected gold nanoparticles. Langmuir. 20:1909-1914.
34. Sorkine, P., H. Nagar, A. Weinbroum, A. Setton, E. Israitel, A. Scarlatt. 1996. Administration of amphotericin B in lipid emulsion decreases nephrotoxicity: results of a prospective, randomized, controlled study in critically ill patients. Crit. Care. Med. 24:1311-1315.
35. Tarasov, V. N., V. K. Boldasov. 1967. Bactericidal and antitoxic effects of synthetic polymers (literature review). Voenno-Meditsinskii Zhurnal. 9:34-40.
36. Tsuji, M., M. Takema, H. Miwa, J. Shimada, and S. Kuwahara. 2003. In vivo antibacterial activity of S-3578, a new broad-spectrum cephalosporin: methicillin-resistant Staphylococcus aureus and Pseudomonas aeruginosa experimental infection models. Antimicrob. Agents and Chemother. 47:2507-2512.
37. Turos, E., G. S. K. Reddy, K. Greenhalgh, P. Ramaraju, S. C. Abeylath, S. Jang, S. Dickey, and D. V. Lim. 2007. Penicillin-bound polyacrylate nanoparticles: Restoring the activity of b-lactam antibiotics against MRSA. Bioorg. Med. Chem. Lett. 17:3468-3472.
38. Turos, E., J.-Y. Shim, Y. Wang, K. Greenhalgh, G. S. K. Reddy, S. Dickey, and D. V. Lim. 2007. Antibiotic-conjugated polyacrylate nanoparticles: New opportunities for development of anti-MRSA agents. Bioorg. Med. Chem. Lett. 17:53-56.
39. van Dardel, 0., D. Mebius, B. Svensson. 1981. Fat emulsion as a vehicle for diazepam: A study of 9,492 patients. Br. J. Anaesth. 55:41-47.
40. Walsh, C. 2003. Where will new antibiotics come from? Nat. Rev. Microbiol. 1:65-70.
41. Washington, C., M. Lance, S. S. Davis. 1993. Toxicity of amphotericin B emulsion formulations. J. Antimicrob. Chemother. 31:806-808.
42. Weigelt, J., K. Itani, D. Stevens, W. Lau, M. Dryden, and C. Knirsch. 2005. Linezolid CSSTI study group. Linezolid versus vancomycin in treatment of complicated skin and soft tissue infections. Antimicrob. Agents. Chemother. 49:2260-2266.
43. Yarkoni, E., H. J. Rapp. 1978. Toxicity of emulsified trehalose-6,6′-dimycolate (cord factor) in mice depends on size distribution of mineral oil droplets. Infect. Immun. 20:856-860.
44. Zafeiris, S. 2004. Antibiotics: A shot in the arm. Nature 431:892-893.
45. Zheng, F., X.-W. Shi, G.-F. Yang, L.-L. Gong, H.-Y. Yuan, Y.-J. Cui, Y. Wang, Y.-M. Du, Y. Li. 2007. Chitosan nanoparticle as gene therapy vector via gastrointestinal mucosa administration: Results of an in vitro and in vivo study. Life Sciences 80:388-396.
46. Petkewich, Rachel. “Science & Technology.” Chemical & Engineering News:: What's That Stuff? Liquid Bandages. American Chemical Society. Web. 13 Mar. 2012. <http://pubs.acs.org/cen/whatstuff/86/8624sci5.html>
47. “wound.” The American Heritage® Science Dictionary. Houghton Mifflin Company. 6 Mar. 2012. <Dictionary.com http://dictionary.reference.com/browse/wound>.
48. Ruszczak, Zbigniew, Robert A. Schwartz, Ewa Joss-Wichman, Rafal Wichman, and Anna Zalewska. “Surgical Dressings.” Ed. Dirk M. Elston. Medscape Reference. WebMD.
49. ABC of wound healing: Wound dressings. Vanessa Jones, Joseph E Grey, Keith G Harding. BMJ 2006; 332:777-780, doi:10.1136/bmj.332.7544.777
50. Ngan, Vanessa. “Synthetic Wound Dressings.” Wound Dressings (synthetic). New Zealand Dermatological Society Incorporated, 2005. Web. 13 Mar. 2012. <http://dermnetnz.org/procedures/dressings.html>.
51. “Nexcare No Sting Liquid Bandage Spray.” 3M US: Nexcare No Sting Liquid Bandage Spray. 3M. Web. 13 Mar. 2012. <http://www.3m.com/product/information/Nexcare-No-Sting-Liquid-Bandage-Spray.html>.
52. Steffansen, Bente, and Sofie P. K. Herping. “Novel Wound Models for Characterizing Ibuprofen Release from Foam Dressings.” International Journal of Pharmaceutics 364.1 (2008): 150-55. Print.
53. Crandall, Mary A. Kalorama Wound Care Markets 2011. Rep. no. KLI6422062. New York: Kalorama Information, 2011. Print.
54. “Biomaterial of the Month.” Hydrogels. Society for Biomaterials, 1 Jun. 2007. Web. 13 Mar. 2012. <http://www.biomaterials.org/week/bio17.cfm>.
55. “Skin Antiseptic Liquid Bandage 30 ml/1fl.oz.” Reviews. Buying Guides & Consumer Product Reviews. Shopping.com Network. Web. 14 Mar. 2012. <http://www.epinions.com/reviews/New_Skin_Antiseptic_Liquid_Bandage_{—}1_Fl_oz?sb=1>.
56. Lawrence, J C., Injury, 1982; 13:500-512

1-13. (canceled)

14. A method of applying a composition to hard or soft tissue of a subject in need thereof, comprising applying the composition to the hard or soft tissue, wherein the composition comprises an emulsion of polymer and water, wherein the polymer comprises a copolymer of a base acrylate and a supporting monomer.

15. The method of claim 14, wherein the composition is applied to protect or cover a wound.

16. The method of claim 14, wherein the composition is applied to adhere and repair injuries to soft tissue.

17. The method of claim 14, wherein the composition is applied as a medical sealant or a medical adhesive.

18. The method of claim 14, wherein the composition is applied as a hemostatic agent.

19. The method of claim 14, wherein the composition is applied as a filler.

20. The method of claim 14, wherein the composition is applied to an absorbent material.

21. The method of claim 14, wherein the composition is applied to deliver an additive to the tissue.

22. The method of claim 14, wherein the polymer is a microemulsion polymerized nanoparticle.

23. The method of claim 22, wherein the nanoparticle comprises a copolymer of repeating units of a base acrylate monomer and a supporting monomer.

24. The method of claim 23, wherein the base acrylate is butyl acrylate, methyl methacrylate, methyl acrylate or ethyl acrylate, and the supporting monomer is methyl methacrylate, methacrylate, styrene, methacrylamide, phenyl acrylate, ethyl acrylate, a combination of two or more of the foregoing, or a bioactive agent modified to include an acrylate moiety.

25. The method of claim 23, wherein the copolymer comprises two or more supporting monomers.

26. The method of claim 14, wherein the copolymer is in the form of nanoparticles ranging from 10-400 nm, and wherein the additive is a bioactive agent that is encapsulated within the nanoparticles.
